SEQUENCE LISTING (9 sequences)

SEQ ID NO: 1 - DNA, 1357 bp, Homo sapiens
  CDS (210)...(1196)
cgcccggcccctggccccagcaccctgtccgctgccgcctcagagccgggaaaagcagccggagcccccgccgcccctgccgcagcgcgggcggtcagcgcgcagcccggcacccgcagcctgcagcctgcagcccgcagcccgcagcccggagccagatcgcgggctcagaccgaacccgactcgaccgccgcccccagccaggcgccatgctgccgcttctgctgggcctgctgggcccagcggcctgctgggccctgggcccgacccccggcccgggatcctctgagctgcgctcggccttctcggcggcacgcaccacccccctggagggcacgtcggagatggcggtgaccttcgacaaggtgtacgtgaacatcgggggcgacttcgatgtggccaccggccagtttcgctgccgcgtgcccggcgcctacttcttctccttcacggctggcaaggccccgcacaagagcctgtcggtgatgctggtgcgaaaccgcgacgaggtgcaggcgctggccttcgacgagcagcggcggccaggcgcgcggcgcgcagccagccagagcgccatgctgcagctcgactacggcgacacagtgtggctgcggctgcatggcgccccgcactacgcgctaggcgcgcccggcgccaccttcagcggctacctagtctacgccgacgccgacgctgacgcgcctgcgcgcgggccgcccgcgccccccgagccgcgctcggccttctcggcggcgcgcacgcgcagcttggtgggctcggacgctggccccgggccgcggcaccaaccactcgccttcgacaccgagttcgtcaacattggcggcgacttcgacgcggcggccggcgtgttccgctgccgtctgcccggcgcctacttcttctccttcacgctgggcaagctgccgcgtaagacgctgtcggttaagctgatgaagaaccgcgacgaggtgcaggccatgatttacgacgacggcgcgtcgcggcgccgcgagatgcagagccagagcgtgatgctggccctgcggcgcggcgacgccgtctggctgctcagccacgaccacgacggctacggcgcctacagcaaccacggcaagtacatcaccttctccggcttcctggtgtaccccgacctcgcccccgccgccccgccgggcctcggggcctcggagctactgtgagccccgggccagagaagagcccgggagggccaggggcgtgcatgccaggccgggcccgaggctcgaaagtcccgcgcgagcgccacggcctccgggcgcgcctggactctgccaataaagcggaaagcgggcacgcgcagcgcccggcagcccaggca

SEQ ID NO: 2 - protein, 329 aa, Homo sapiens
MLPLLLGLLGPAACWALGPTPGPGSSELRSAFSAARTTPLEGTSEMAVTFDKVYVNIGGDFDVATGQFRCRVPGAYFFSFTAGKAPHKSLSVMLVRNRDEVQALAFDEQRRPGARRAASQSAMLQLDYGDTVWLRLHGAPHYALGAPGATFSGYLVYADADADAPARGPPAPPEPRSAFSAARTRSLVGSDAGPGPRHQPLAFDTEFVNIGGDFDAAAGVFRCRLPGAYFFSFTLGKLPRKTLSVKLMKNRDEVQAMIYDDGASRRREMQSQSVMLALRRGDAVWLLSHDHDGYGAYSNHGKYITFSGFLVYPDLAPAAPPGLGASELL

SEQ ID NO: 3 - protein, 31 aa, Artificial Sequence
  C1q Aromatic Motif
FXXXXXXXXXXXXXXXXXFXXXXXGXYXFXX

SEQ ID NO: 4 - DNA, 987 bp, Artificial Sequence
  Degenerate nucleotide sequence encoding the polypeptide of SEQ ID NO2
atgytnccnytnytnytnggnytnytnggnccngcngcntgytgggcnytnggnccnacnccnggnccnggnwsnwsngarytnmgnwsngcnttywsngcngcnmgnacnacnccnytngarggnacnwsngaratggcngtnacnttygayaargtntaygtnaayathggnggngayttygaygtngcnacnggncarttymgntgymgngtnccnggngcntayttyttywsnttyacngcnggnaargcnccncayaarwsnytnwsngtnatgytngtnmgnaaymgngaygargtncargcnytngcnttygaygarcarmgnmgnccnggngcnmgnmgngcngcnwsncarwsngcnatgytncarytngaytayggngayacngtntggytnmgnytncayggngcnccncaytaygcnytnggngcnccnggngcnacnttywsnggntayytngtntaygcngaygcngaygcngaygcnccngcnmgnggnccnccngcnccnccngarccnmgnwsngcnttywsngcngcnmgnacnmgnwsnytngtnggnwsngaygcnggnccnggnccnmgncaycarccnytngcnttygayacngarttygtnaayathggnggngayttygaygcngcngcnggngtnttymgntgymgnytnccnggngcntayttyttywsnttyacnytnggnaarytnccnmgnaaracnytnwsngtnaarytnatgaaraaymgngaygargtncargcnatgathtaygaygayggngcnwsnmgnmgnmgngaratgcarwsncarwsngtnatgytngcnytnmgnmgnggngaygcngtntggytnytnwsncaygaycaygayggntayggngcntaywsnaaycayggnaartayathacnttywsnggnttyytngtntayccngayytngcnccngcngcnccnccnggnytnggngcnwsngarytnytn

SEQ ID NO: 5 - DNA, 24 bp, Artificial Sequence
  Oligonucleotide ZC20,839
atgtacttgccgtggttgctgtag

SEQ ID NO: 6 - DNA, 23 bp, Artificial Sequence
  Oligonucleotide ZC20840
cgacaccgagttcgtcaacattg

SEQ ID NO: 7 - DNA, 325 bp, Artificial Sequence
  Degenerate nucleotide sequence encoding the polypeptide of SEQ ID NO2.
ctggccccgggccgcggcaccaaccactcgccttcgacaccgagttcgtcaacattggcggcgacttcgacgcggcggccggcgtgttccgctgccgtctgnccggcgcctacttcttctncttcacgctgggcaagctgccgcgtaagacgctgtcggttaagctgatgaagaaccgcgacgaggtgcaggccatgatttacgacgacggcgcgtcgcggcgccgcgagatgcagagccagagcgtgatgctggccctgcggcgcggngacgccgtctggctgtcagccacgaccacgacggctacggcgcctacagcaaccac

SEQ ID NO: 8 - DNA, 18 bp, Artificial Sequence
  Oligonucleotide ZC22162
ccgcggcaccaaccactc

SEQ ID NO: 9 - DNA, 18 bp, Artificial Sequence
  Oligonucleotide ZC 22168
gtcgcggttcttcatcag

(19) United States
(12) Patent Application Publication
(10) Pub. No.: US 2003/0170781 A1
(43) Pub. Date: Sep. 11, 2003

(54) Secreted protein zacrp4

(75) Inventors: James L. Holloway, Seattle, WA (US); Si Lok, Seattle, WA (US)

Correspondence Address: Brian J. Walsh, Patent Department, ZymoGenetics, Inc., 1201 Eastlake Avenue East, Seattle, WA, 98102, US

(73) Assignee: ZymoGenetics, Inc.

(21) Appl. No.: 10/085,167

(22) Filed: Feb. 27, 2002

Related U.S. Application Data
(63) Continuation of application No. 09/606,031, filed on Jun. 28, 2000, now abandoned.
(60) Provisional application No. 60/141,928, filed on Jul. 1, 1999.

(51) Int. Cl. C07H 21/04; C12N 9/00; C12P 21/02; C12N 5/06

(52) U.S. Cl. 435/69.1; 435/183; 435/320.1; 435/325; 536/23.2

(57) ABSTRACT

The present invention relates to polynucleotide and polypeptide molecules for zacrp4, a secreted protein having tandem C1q globular domains. Zacrp4 is highly expressed in neuronal and reproductive tissues and may be used in the study cell-cell communication and the regulation of cellular processes therein. The present invention also includes antibodies to the zacrp4 polypeptides.

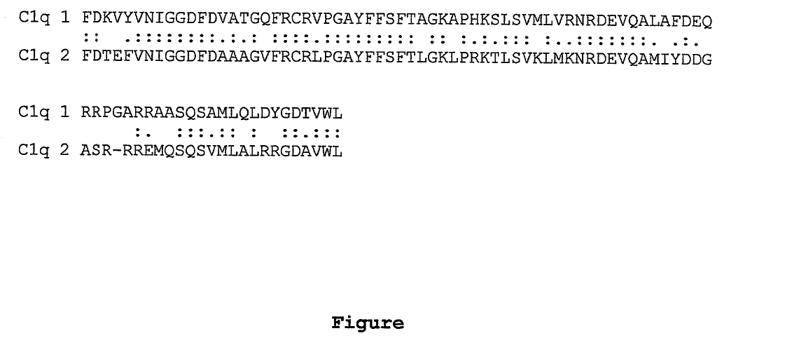

### REFERENCE TO RELATED APPLICATIONS

This application is related to Provisional Application No. 60/141,928, filed on Jul. 1, 1999. Under 35 U.S.C. §119(e)(1), this application claims benefit of said Provisional Application.

### BACKGROUND OF THE INVENTION

Complement factor C1q, a subunit of the C1 enzyme complex, consists of six copies of three related polypeptides or subunits (A, B and C chains), with each polypeptide being about 225 amino acids long with a near amino-terminal collagen domain comprised of collagen repeats having the sequence of Gly-Xaa-Pro or Gly-Xaa-Xaa and a carboxy-terminal globular C1q domain made up of ten beta strands. Six triple helical regions are formed by the collagen domains of the six A, six B and six C chains, forming a central region and six stalks. A globular head portion is formed by association of the globular carboxy terminal domain of an A, a B and a C chain. C1q is therefore composed of six globular heads linked via six collagen-like stalks to a central fibril region. Sellar et al., Biochem. J. 274: 481-90, 1991. This configuration is often referred to as a bouquet of flowers.

A single C-terminal C1q domain has been reported in several other collagen-like proteins, including zsig37 (WO 99104000), zsig39 (WO 99/10492), chipmunk hibernation-associated plasma proteins HP-20, HP-25 and HP-21 (Takamatsu et al., Mol. Cell. Biol. 13: 1516-21, 1993 and Kondo & Kondo, J. Biol. Chem. 267: 473-8, 1992), human precerebellin (Urade et al., Proc. Natl. Acad. Sci. USA 88:1069-73, 1991), human endothelial cell multimerin (Hayward et al., J. Biol. Chem. 270:18246-51, 1995), vertebrate collagens type VIII and X (Muragaki et al., Eur. J. Biochem. 197:615-22, 1991) and ACRP30 (Scherer et al., J. Biol. Chem. 270(45): 26746-9, 1995). The structural elements such as folding topologies, conserved residues and similar trimer interfaces and intron positions of the C1q domain of ACRP30 are homologous to the tumor necrosis factor family suggesting a link between the TNF and C1q families.

C1q has been found to stimulate defense mechanisms as well as trigger the generation of toxic oxygen species that can cause tissue damage (Tenner, Behring Inst. Mitt. 93:241-53, 1993). C1q binding sites are found on platelets. Additionally complement and C1q play a role in inflammation. The complement activation is initiated by binding of C1q to immunoglobulins. Inhibitors of C1q and the complement pathway would be useful for anti-inflammatory applications, inhibition of complement activation.

The present invention provides such polypeptides for these and other uses that should be apparent to those skilled in the art from the teachings herein.

### SUMMARY OF THE INVENTION

Within one aspect the invention provides an isolated polypeptide comprising: a first C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2, and a cysteine residue corresponding to residue 70 of SEQ ID NO:2; and a second C1q domain joined to the carboxy terminal of said first C1q domain, said second C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2, and a cysteine residue corresponding to residue 223, a glycine residue corresponding to residue 228 of SEQ ID NO:2. Within one embodiment the polypeptide further comprises a secretory signal sequence. Within a related embodiment the secretory signal sequence comprises amino acid residues 1-16 of SEQ ID NO:2. The invention also provides an isolated polypeptide comprising a sequence of amino acid residues that is at least 80% identical in amino acid sequence to residues 17 to 329 of SEQ ID NO:2, wherein the sequence comprises: a first C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2, and a cysteine residue corresponding to residue 70 of SEQ ID NO:2; and a second C1q domain joined to the carboxy terminal of the first C1q domain, the second C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2, and a cysteine residue corresponding to residue 223, a glycine residue corresponding to residue 228 of SEQ ID NO:2. Within one embodiment the polypeptide is at least 90% identical in amino acid sequence to residues 17 to 329 of SEQ ID NO:2. Within another embodiment the amino acid percent identity is determined using a FASTA program with ktup=1, gap opening penalty=10, gap extension penalty=1, and substitution matrix=blosum62, with other parameters set as default. Within another embodiment any differences between the polypeptide and SEQ ID NO:2 are due to conservative amino acid substitutions. Within a further embodiment the polypeptide specifically binds with an antibody that specifically binds with a polypeptide consisting of the amino acid sequence of SEQ ID NO:2. Within still another embodiment the first C1q domain comprises amino acid residues 17-159 of SEQ ID NO:2. Another embodiment provides that the second C1q domain comprises amino acid residues 160-328 of SEQ ID NO:2. Still another embodiment provides that the polypeptide comprises residues 17-328 of SEQ ID NO:2. Another embodiment provides a polypeptide as described above covalently linked at the amino or carboxyl terminus to a moiety selected from the group consisting of affinity tags, toxins, radionucleotides, enzymes and fluorophores.

The invention also provides an isolated polypeptide comprising: a signal sequence, a first C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2 and a cysteine residue corresponding to amino acid residue 70 of SEQ ID NO:2; and a second C1q domain joined to the carboxy terminal of the first C1q domain, the second C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2 and a cysteine residue corresponding to residue 223 of SEQ ID NO:2; wherein the polypeptide specifically binds with an antibody that specifically binds with a polypeptide consisting of the amino acid sequence of SEQ ID NO:2.

Also provides is an isolated polypeptide selected from the group consisting of: a) a polypeptide consisting of amino acid residue 17 to amino acid residue 159 of SEQ ID NO:2 or b) a polypeptide consisting of amino acid residue 160 to amino acid residue 329 of SEQ ID NO:2.

The invention also provides a fusion protein consisting essentially of a first portion and a second portion joined by a peptide bond, the first portion consisting of a polypeptide selected from the group consisting of: a) polypeptide as described above; b) a polypeptide comprising the amino acid sequence of residues 17-159 of SEQ ID NO:2; c) a polypeptide comprising the amino acid sequence of residues 160-329 of SEQ ID NO:2; and d) a polypeptide comprising the amino acid sequence of residues 17-329 of SEQ ID NO:2; and the second portion comprising another polypeptide.

Also provides is a polypeptide as described above in combination with a pharmaceutically acceptable vehicle.

Within another aspect the invention provides a method of producing an antibody to a polypeptide comprising: inoculating an animal with a polypeptide selected from the group consisting of: a) polypeptide as described above; b) a polypeptide comprising the amino acid sequence of residues 17-159 of SEQ ID NO:2; c) a polypeptide comprising the amino acid sequence of residues 160-329 of SEQ ID NO:2; d) a polypeptide comprising the amino acid sequence of residues 17-329 of SEQ ID NO:2; and e) a polypeptide or polypeptide fragment of SEQ ID NO:2; and wherein the polypeptide elicits an immune response in the animal to produce the antibody; and isolating the antibody from the animal.

Also provided is an antibody or antibody fragment that specifically binds to a polypeptide as described above. Within one embodiment the antibody is selected from the group consisting of: a) polyclonal antibody; b) murine monoclonal antibody; c) humanized antibody derived from b); and d) human monoclonal antibody. Within another embodiment the antibody fragment is selected from the group consisting of F(ab′), F(ab), Fab′, Fab, Fv, scFv, and minimal recognition unit. Within still another embodiment is provided an anti-idiotype antibody that specifically binds to the antibody described above. Also provided by the invention is a binding protein that specifically binds to an epitope of a polypeptide as described above.

Within another aspect of the invention is provided an isolated polynucleotide molecule encoding a polypeptide as described above. The invention also provides an isolated polynucleotide molecule encoding a polypeptide comprising a sequence of amino acid residues that is at least 80% identical in amino acid sequence to residues 17 to 329 of SEQ ID NO:2, wherein the sequence comprises: a first C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2, and a cysteine residue corresponding to residue 70 of SEQ ID NO:2; and a second C1q domain joined to the carboxy terminal of the first C1q domain, the second C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2, and a cysteine residue corresponding to residue 223, a glycine residue corresponding to residue 228 of SEQ ID NO:2. Within one embodiment the polypeptide is at least 90% identical in amino acid sequence to residues 17-329 of SEQ ID NO:2. Within another embodiment the amino acid percent identity is determined using a FASTA program with ktup=1, gap opening penalty=10, gap extension penalty=1, and substitution matrix=blosum62, with other parameters set as default. Within still another embodiment the polypeptide further comprises a secretory signal sequence. Within a related embodiment the secretory signal sequence comprises amino acid residues 1-16 of SEQ ID NO:2. Within another embodiment differences between the polypeptide and SEQ ID NO:2 are due to conservative amino acid substitutions. Within still another embodiment the polypeptide specifically binds with an antibody that specifically binds with a polypeptide consisting of the amino acid sequence of SEQ ID NO:2. Within another embodiment the polynucleotide molecule remains hybridized following stringent wash conditions to a polynucleotide consisting of the nucleotide sequence of SEQ ID NO:1, or the complement of SEQ ID NO:1. Another embodiment provides that the first C1q domain comprises amino acid residues 17-159 of SEQ ID NO:2. Also provided is a polynucleotide wherein the second C1q domain comprises amino acid residues 160-329 of SEQ ID NO:2. Within another embodiment the polypeptide comprises residues 17-329 of SEQ ID NO:2.

Also provided by the invention is an isolated polynucleotide molecule encoding a polypeptide comprising: a signal sequence, a first carboxyl-terminal C1q domain comprising a sequence of SEQ ID NO:3, 10 beta strands corresponding to amino acid residues 31-35, 52-54, 60-63, 67-69, 73-84, 89-95, 101-108, 11.2-126, 131-136 and 150-154 of SEQ ID NO:2 and a cysteine residue corresponding to amino acid residue 70 of SEQ ID NO:2; and a second carboxyl-terminal C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2 and a cysteine residue corresponding to residue 223 of SEQ ID NO:2; wherein the polypeptide specifically binds with an antibody that specifically binds with a polypeptide consisting of the amino acid sequence of SEQ ID NO:2, and the polynucleotide molecule remains hybridized following stringent wash conditions to a polynucleotide consisting of the nucleotide sequence of SEQ ID NO:1, or the complement of SEQ ID NO: 1.

The invention further provides an isolated polynucleotide molecule selected from the group consisting of, a) a sequence of nucleotides from nucleotide 1 to nucleotide 1357 of SEQ ID NO:1; b) a contiguous sequence of nucleotides from nucleotide 210 to nucleotide 1196 of SEQ ID NO:1; c) a contiguous sequence of nucleotides from nucleotide 258 to nucleotide 1196 of SEQ ID NO: 1; d) a contiguous sequence of nucleotides from nucleotide 258 to nucleotide 686 of SEQ ID NO: 1; e) a contiguous polynucleotide encoding a polypeptide consisting of the sequence of amino acid residues 17 to 159 of SEQ ID NO:2; f) a polynucleotide encoding a polypeptide consisting of the sequence of amino acid residues 160 to 329 of SEQ ID NO:2; g) a polynucleotide encoding a polypeptide consisting of the sequence of amino acid residues 17 to 329 of SEQ ID NO:2; h) a polynucleotide that remains hybridized following stringent wash conditions to a polynucleotide consisting of the nucleotide sequence of SEQ ID NO:1, or the complement of SEQ ID NO:1; i) the nucleotide sequences complementary to a), b), c), d), e), f), g) or h) and m) degenerate nucleotide sequences of e), f) or g).

The invention also provides an isolated polynucleotide molecule encoding a fusion protein consisting essentially of a first portion and a second portion joined by a peptide bond, the first portion is selected from the group consisting of: a) polypeptide according to claim 1; b) a polypeptide comprising the amino acid sequence of residues 17-159 of SEQ ID NO:2; c) a polypeptide comprising the amino acid sequence of residues 160-329 of SEQ ID NO:2; and d) a polypeptide comprising the amino acid sequence of residues 17-329 of SEQ ID NO:2; and the second portion comprising another polypeptide.

Also provided is an isolated polynucleotide molecule consisting of the sequence of nucleotide 1 to nucleotide 987 of SEQ ID NO:4.

Within another aspect the invention provides an expression vector comprising the following operably linked elements: a transcription promoter; a DNA segment encoding a polypeptide as described above; and a transcription terminator. Within one embodiment the DNA segment encodes a polypeptide covalently linked at the amino or carboxyl terminus to an affinity tag. The invention also provides that the DNA segment further encodes a secretory signal sequence operably linked to the polypeptide. Within a related embodiment the secretory signal sequence comprises residues 1-16 of SEQ ID NO:2.

Within another aspect of the invention is a cultured cell into which has been introduced an expression vector as described above, wherein the cell expresses the polypeptide encoded by the DNA segment.

Also provided is a method of producing a protein comprising: culturing a cell into which has been introduced an expression vector as described above; whereby the cell expresses the polypeptide encoded by the DNA segment; and recovering the expressed protein.

### BRIEF DESCRIPTION OF THE DRAWING

The FIGURE illustrates an alignment of the aromatic motifs within the first and second C1q domains of zacrp4. The aromatic motif within the first C1q domain (C1q1) is from amino acid residue 50 to amino acid residue 80 of SEQ ID NO:2. The aromatic motif within the second C1q domain (C1q2) is from amino acid residue 203 to amino acid residue 233 of SEQ ID NO:2.

### DETAILED DESCRIPTION OF THE INVENTION

Prior to setting forth the invention in detail, it may be helpful to the understanding thereof to define the following terms.

The term “affinity tag” is used herein to denote a peptide segment that can be attached to a polypeptide to provide for purification or detection of the polypeptide or provide sites for attachment of the polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), substance P, Flag™ peptide (Hopp et al., Biotechnology 6:1204-10, 1988; available from Eastman Kodak Co., New Haven, Conn.), streptavidin binding peptide, or other antigenic epitope or binding domain. See, in general Ford et al., Protein Expression and Purification 2: 95-107, 1991. DNAs encoding affinity tags are available from commercial suppliers (e.g., Pharmacia Biotech, Piscataway, N.J.).

The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides and proteins. Where the context allows, these terms are used with reference to a particular sequence or portion of a polypeptide or protein to denote proximity or relative position. For example, a certain sequence positioned carboxyl-terminal to a reference sequence within a protein is located proximal to the carboxyl terminus of the reference sequence, but is not necessarily at the carboxyl terminus of the complete protein.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGCTTAGCTT-3′ are 5′-TAGCTTgagtct-3′ and 3′-gtcgacTACCGA-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” denotes a DNA molecule, linear or circular, that comprises a segment encoding a polypeptide of interest operably linked to additional segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, denotes that the segments are arranged so that they function in concert for their intended purposes, e.g. transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned, cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nt, more preferably 20-30 nt. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

The term “promoter” denotes a portion of a gene containing DNA sequences that provide for the binding of RNA polymerase and initiation of transcription. Promoter sequences are commonly, but not always, found in the 5′ non-coding regions of genes.

The term “receptor” denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. Most nuclear receptors also exhibit a multi-domain structure, including an amino-terminal, transactivating domain, a DNA binding domain and a ligand binding domain. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger peptide is commonly cleaved to remove the secretory peptide during transit through the secretory pathway.

A “soluble receptor” is a receptor polypeptide that is not bound to a cell membrane. Soluble receptors are most commonly ligand-binding receptor polypeptides that lack transmembrane and cytoplasmic domains. Soluble receptors can comprise additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis or translated from alternatively spliced mRNAs. Receptor polypeptides are said to be substantially free of transmembrane and intracellular polypeptide segments when they lack sufficient portions of these segments to provide membrane anchoring or signal transduction, respectively.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±110%.

All references cited herein are incorporated by reference in their entirety.

The present invention is based in part upon the discovery of a novel DNA sequence that encodes a polypeptide having regions of homology to complement factor C1q. The novel DNA sequence encodes a polypeptide having an amino-terminal signal sequence (residues 1-16 of SEQ ID NO:2) and two tandem C1q domains (residues 17-159 and 160-328 of SEQ ID NO:2). An alignment of the two C1q domains (see FIGURE) shows that they share a conserved cysteine residue, residues 70 and 223 of SEQ ID NO:2. Such a conserved cysteine residue is seen in other proteins having a C1q domain, such as ACRP30. There is a fair amount of conserved structure within the C1q domains to enable proper folding. An aromatic motif (F-X(5)-[ND]-X(4)-[FYWL]-X(6)-F-X(5)-G-X-Y-X-F-X-[Y] (SEQ ID NO:3) is also found within both C1q domains, residues 50-80 and 203-233 of SEQ ID NO:2. X represents any amino acid residue and the number in parentheses ( ) indicates the number of amino acid residues. The amino acid residues contained within the square parentheses [ ] restrict the choice of amino acid residues at that particular position. Also, the zacrp4 polypeptides of the present invention include potential phosphorylation sites at residues 38, 244 and 264 of SEQ ID NO:2. Potential myristylation sites are found at residues 66, 113, 145, 148, 219, 295 and 322 of SEQ ID NO:2. Potential prenylation sites are found in association with the conserved cysteine residues at residue 70 and 223 of SEQ ID NO:2.

The globular C1q domain of ACRP30 has been determined to have a 10 beta strand “jelly roll” topology (Shapiro and Scherer, Curr. Biol. 8:335-8, 1998) that shows significant homology to the TNF family. The two C1q domains of zacrp4 each contain all 10 beta-strands of this structure. Within the first C1q domain the strands comprise amino acid residues 31-35, 52-54, 60-63, 67-69, 73-84, 89-95, 101-108, 112-126, 131-136 and 150-154 of SEQ ID NO:2. Within the second C1q domain the strands comprise amino acid residues 179-183, 206-208, 214-217, 221-223, 227-238, 243-249, 254-262, 267-278, 283-288 and 305-309 of SEQ ID NO:2. These strands have been designated “A”, “A′”, “B”, “B′”, “C”, “D”, “E”, “F”, “G” and “H” respectively.

The C1q domains of zacrp4 each have two receptor binding loops. Within the first C1q domain these loops are found at amino acid residues 37-62 and 94-108 of SEQ ID NO:2. Within the second C1q domain they are found at amino acid residues 185-216 and 248-262 of SEQ ID NO:2. Amino acid residues 74 (Gly), 76 (Tyr), 126 (Leu) and 151 (Phe) of SEQ ID NO:2 in the first C1q domain and 228 (Gly), 230 (Tyr), 279 (Leu) and 307 (Phe) of SEQ ID NO:2 within the second C1q domain appear to be conserved across the superfamily including CD40, TNFα, TNFβ, ACRP30 and zacrp4. Those skilled in the art will recognize that these boundaries are approximate, and are based on alignments with known proteins and predictions of protein folding.

The conserved amino acid residues and motifs in the C1q domains of the zacrp4 polypeptide can be used as a tool to identify new family members. For instance, reverse transcription-polymerase chain reaction (RT-PCR) can be used to amplify sequences encoding the conserved motifs from RNA obtained from a variety of tissue sources. In particular, highly degenerate primers designed from conserved sequences described herein are useful for this purpose.

Analysis of the tissue distribution of the mRNA corresponding to this novel DNA using a full length probe showed that zacrp4 is strongly represented in brain, spinal cord, ovary and testis. Zacrp4 is also expressed in adrenal gland, bone marrow, thyroid, liver, prostate, small intestine and colon.

The novel zacrp4 polypeptides of the present invention were initially identified by querying an EST database for sequences having homology with adipocyte-specific protein homolog zsig37 (WO99/04000). Polypeptides corresponding to ESTs meeting those search criteria were compared to known sequences to identify proteins having homology to known C1q domain containing proteins. The corresponding full length cDNA was identified as described herein. The resulting 1357 bp sequence is disclosed in SEQ ID NO:1. Zacrp4 shares 35% identity at the amino acid level with the C1q domain of murine ACRP30 and 32% with human ACRP30.

The full nucleotide sequence encoding the zacrp4 polypeptide of SEQ ID NO:2 was identified from a clone obtained from a total fetus library. Other libraries that might also be searched for such clones include pituitary, brain, spinal cord, ovary, testis and the like.

Another aspect of the present invention includes zacrp4 polypeptide fragments. Preferred fragments include those containing independent C1q domains of zacrp4 polypeptides, ranging from amino acid 17-159 or 160-328 of SEQ ID NO:2, a portion of the zacrp4 polypeptide containing a C1q domain or an active portion of the C1q domain. These fragments are particularly useful in the study or modulation of cell to cell communication, neurotransmission, anti-microbial activity may also be present in such fragments.

The present invention also provides polynucleotide molecules, including DNA and RNA molecules, that encode the zacrp4 polypeptides disclosed herein. Those skilled in the art will readily recognize that, in view of the degeneracy of the genetic code, considerable sequence variation is possible among these polynucleotide molecules. SEQ ID NO:4 is a degenerate DNA sequence that encompasses all DNAs that encode the zacrp4 polypeptide of SEQ ID NO:2. Those skilled in the art will recognize that the degenerate sequence of SEQ ID NO:4 also provides all RNA sequences encoding SEQ ID NO:2 by substituting U for T. Thus, zacrp4 polypeptide-encoding polynucleotides comprising nucleotide 1 to nucleotide 987 of SEQ ID NO:4 and their RNA equivalents are contemplated by the present invention. Table 1 sets forth the one-letter codes used within SEQ ID NO:4 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolutions | Complement | Resolutions |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:4, encompassing all possible codons for a given amino acid, are set forth in Table 2.

TABLE 3

| | One- | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | CAN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAG GAT | GAY |
| Glu | E | GAA GAG | GAR |
| Gln | Q | CAA GAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAG TAT | TAY |
| Trp | W | TGG | TGG |
| Ter | • | TAA TAG TGA | TRR |
| Asn\|Asp | B | | RAY |
| Glu\|Gln | Z | | SAR |
| Any | X | | NNN |
| Gap | - | --- | |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids 115 Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:4 serves as a template for optimizing expression of polynucleotides in various cell types and species commonly used in the art and disclosed herein. Sequences containing preferential codons can be tested and optimized for expression in various species, and tested for functionality as disclosed herein.

The present invention further provides variant polypeptides and nucleic acid molecules that represent counterparts from other species (orthologs). These species include, but are not limited to mammalian, avian, amphibian, reptile, fish, insect and other vertebrate and invertebrate species. Of particular interest are zacrp4 polypeptides from other mammalian species, including murine, porcine, ovine, bovine, canine, feline, equine, and other primate polypeptides. Orthologs of human zacrp4 can be cloned using information and compositions provided by the present invention in combination with conventional cloning techniques. For example, a cDNA can be cloned using mRNA obtained from a tissue or cell type that expresses zacrp4 as disclosed herein. Suitable sources of mRNA can be identified by probing northern blots with probes designed from the sequences disclosed herein. A library is then prepared from mRNA of a positive tissue or cell line.

A zacrp4-encoding cDNA can then be isolated by a variety of methods, such as by probing with a complete or partial human cDNA or with one or more sets of degenerate probes based on the disclosed sequences. A cDNA can also be cloned using the polymerase chain reaction with primers designed from the representative human zacrp4 sequences disclosed herein. Within an additional method, the cDNA library can be used to transform or transfect host cells, and expression of the cDNA of interest can be detected with an antibody to zacrp4 polypeptide. Similar techniques can also be applied to the isolation of genomic clones.

Those skilled in the art will recognize that the sequence disclosed in SEQ ID NO:1 represents a single allele of human zacrp4, and that allelic variation and alternative splicing are expected to occur. Allelic variants of this sequence can be cloned by probing cDNA or genomic libraries from different individuals according to standard procedures. Allelic variants of the nucleotide sequence shown in SEQ ID NO:1, including those containing silent mutations and those in which mutations result in amino acid sequence changes, are within the scope of the present invention, as are proteins which are allelic variants of SEQ ID NO:2. cDNA molecules generated from alternatively spliced mRNAs, which retain the properties of the zacrp4 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Within preferred embodiments of the invention, the isolated nucleic acid molecules can hybridize under stringent conditions to nucleic acid molecules having the nucleotide sequence of SEQ ID NO:1 or to nucleic acid molecules having a nucleotide sequence complementary to SEQ ID NO: 1. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions include temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software, such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ.

For smaller probes, <50 base pairs, hybridization is typically carried out at the Tₘ or 5-10° C. below. This allows for the maximum rate of hybridization for DNA-DNA and DNA-RNA hybrids.

The length of the polynucleotide sequence influences the rate and stability of hybrid formation. Smaller probe sequences, <50 base pairs, reach equilibrium with complementary sequences rapidly, but may form less stable hybrids.

Incubation times of anywhere from minutes to hours can be used to achieve hybrid formation. Longer probe sequences come to equilibrium more slowly, but form more stable complexes even at lower temperatures. Incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of polynucleotide sequence will effect the thermal stability of the hybrid complex, thereby influencing the choice of hybridization temperature and the ionic strength of the hybridization buffer. A-T pairs are less stable than G-C pairs in aqueous solutions containing sodium chloride. Therefore, the higher the G-C content, the more stable the hybrid. Even distribution of G and C residues within the sequence also contribute positively to hybrid stability. In addition, the base pair composition can be manipulated to alter the Tₘ of a given sequence. For example, 5-methyldeoxycytidine can be substituted for deoxycytidine and 5-bromodeoxuridine can be substituted for thymidine to increase the Tₘ, whereas 7-deazz-2′-deoxyguanosine can be substituted for guanosine to reduce dependence on Tₘ.

The ionic concentration of the hybridization buffer also affects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1×SSC: 0.15 M sodium chloride, 15 mM sodium citrate) or SSPE (1×SSPE: 1.8 M NaCl, 10 mM NaH₂PO₄, 1 mM EDTA, pH 7.7). By decreasing the ionic concentration of the buffer, the stringency of the hybridization is increased. Typically, hybridization buffers contain from between 10 mM-1 M Na⁺. The addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As an illustration, a nucleic acid molecule encoding a variant zacrp4 polypeptide can be hybridized with a nucleic acid molecule having the nucleotide sequence of SEQ ID NO:1 at 42° C. overnight in a solution comprising 50% formamide, 5×SSC (1×SSC: 0.15 M sodium chloride and 15 mM sodium citrate), 50 mM sodium phosphate (pH 7.6), 5× Denhardt's solution (100× Denhardt's solution: 2% (w/v) Ficoll 400, 2% (w/v) polyvinyl-pyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formamide. Moreover, premixed hybridization solutions are available (e.g., EXPRESSHYB Hybridization Solution from CLONTECH Laboratories, Inc.), and hybridization can be performed according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding a variant zacrp4 polypeptide hybridize with a nucleic acid molecule having the nucleotide sequence of SEQ ID NO:1 (or its complement) under stringent washing conditions, in which the wash stringency is equivalent to 0.5×-2×SSC with 0.1% SDS at 55-65° C., including 0.5×SSC with 0.1% SDS at 55° C., or 2×SSC with 0.1% SDS at 65° C. One of skill in the art can readily devise equivalent conditions, for example, by substituting SSPE for SSC in the wash solution.

Typical highly stringent washing conditions include washing in a solution of 0.1×-0.2×SSC with 0.1% sodium dodecyl sulfate (SDS) at 50-65° C. In other words, nucleic acid molecules encoding a variant zacrp4 polypeptide hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C., including 0.1×SSC with 0.1% SDS at 50° C., or 0.2×SSC with 0.1% SDS at 65° C.

The present invention also provides isolated zacrp4 polypeptides that have a substantially similar sequence identity to the polypeptides of SEQ ID NO:2, or their orthologs. The term “substantially similar sequence identity” is used herein to denote polypeptides having at least 70%, at least 80%, at least 90%, at least 95% or greater than 95% sequence identity to the sequences shown in SEQ ID NO:2, or their orthologs. The present invention further includes nucleic acid molecules that encode such polypeptide variants.

Such nucleic acid molecules can be identified using two criteria: a determination of the similarity between the encoded polypeptide with the amino acid sequence of SEQ ID NO:2, and a hybridization assay, as described herein. Such zacrp4 sequences include nucleic acid molecules (1) that hybridize with a nucleic acid molecule having the nucleotide sequence of SEQ ID NO: 1 (or its complement) under stringent washing conditions, in which the wash stringency is equivalent to 0.5×-2×SSC with 0.1% SDS at 55-65° C., and (2) that encode a polypeptide having at least 70%, at least 80%, at least 90%, at least 95% or greater than 95% sequence identity to the amino acid sequence of SEQ ID NO:2. Alternatively, zacrp4 sequence variants can be characterized as nucleic acid molecules (1) that hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C., and (2) that encode a polypeptide having at least 70%, at least 80%, at least 90%, at least 95% or greater than 95% sequence identity to the amino acid sequence of SEQ ID NO:2.

Percent sequence identity is determined by conventional methods. See, for example, Altschul et al., Bull. Math. Bio. 48:603, 1986, and Henikoff and Henikoff, Proc. Natl. Acad. Sci. USA 89:10915, 1992. Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 3 (amino acids are indicated by the standard one-letter codes). The percent identity is then calculated as: ([Total number of identical matches]/[length of the longer sequence plus the number of gaps introduced into the longer sequence in order to align the two sequences])(100).

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zacrp4. The FASTA algorithm is described by Pearson and Lipman, Proc. Nat. Acad. Sci. USA 85:2444, 1988, and by Pearson, Meth. Enzymol. 183:63, 1990.

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then re-scored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63, 1990.

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

The present invention includes nucleic acid molecules that encode a polypeptide having one or more “conservative amino acid substitutions,” compared with the amino acid sequence of SEQ ID NO:2. Conservative amino acid substitutions can be based upon the chemical properties of the amino acids. That is, variants can be obtained that contain one or more amino acid substitutions of SEQ ID NO:2, in which an alkyl amino acid is substituted for an alkyl amino acid in a zacrp4 amino acid sequence, an aromatic amino acid is substituted for an aromatic amino acid in a zacrp4 amino acid sequence, a sulfur-containing amino acid is substituted for a sulfur-containing amino acid in a zacrp4 amino acid sequence, a hydroxy-containing amino acid is substituted for a hydroxy-containing amino acid in a zacrp4 amino acid sequence, an acidic amino acid is substituted for an acidic amino acid in a zacrp4 amino acid sequence, a basic amino acid is substituted for a basic amino acid in a zacrp4 amino acid sequence, or a dibasic monocarboxylic amino acid is substituted for a dibasic monocarboxylic amino acid in a zacrp4 amino acid sequence.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

The BLOSUM62 table is an amino acid substitution matrix derived from about 2,000 local multiple alignments of protein sequence segments, representing highly conserved regions of more than 500 groups of related proteins (Henikoff and Henikoff, Proc. Natl. Acad. Sci. USA 89:10915, 1992). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Conservative amino acid changes in a zacrp4 gene can be introduced by substituting nucleotides for the nucleotides recited in SEQ ID NO: 1. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)). The ability of such variants to promote the neuronal, anti-microbial, regulatory or other properties of the wild-type protein can be determined using a standard methods, such as the assays described herein. Alternatively, a variant zacrp4 polypeptide can be identified by the ability to specifically bind anti-zacrp4 antibodies.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxyethylcysteine, hydroxyethylhomocysteine, nitroglutamine, homo-glutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenyl-alanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722, 1991, Ellman et al., Methods Enzymol. 202:301, 1991, Chung et al., Science 259:806, 1993, and Chung et al., Proc. Nat. Acad. Sci. USA 90:10145, 1993.

In a second method, translation is carried out in Xenopus oocytes by microinjection of mutated mRNA and chemically aminoacylated suppressor tRNAs (Turcatti et al., J. Biol. Chem. 271:19991, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395, 1993).

A limited number of non-conservative amino acids, amino acids that are not encoded by the genetic code, non-naturally occurring amino acids, and unnatural amino acids may be substituted for zacrp4 amino acid residues.

Multiple amino acid substitutions can be made and tested using known methods of mutagenesis and screening, such as those disclosed by Reidhaar-Olson and Sauer (Science 241:53, 1988) or Bowie and Sauer (Proc. Nat. Acad. Sci. USA 86:2152, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832, 1991, Ladner et al., U.S. Pat. No. 5,223,409, Huse, international publication No. WO 92/06204, and region-directed mutagenesis (Derbyshire et al., Gene 46:145, 1986, and Ner et al., DNA 7:127, 1988).

Variants of the disclosed zacrp4 nucleotide and polypeptide sequences can also be generated through DNA shuffling as disclosed by Stemmer, Nature 370:389, 1994, Stemmer, Proc. Nat. Acad. Sci. USA 91:10747, 1994, and international publication No. WO 97/20078. Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode biologically active polypeptides, or polypeptides that bind with anti-zacrp4 antibodies, can be recovered from the host cells and rapidly sequenced using modem equipment. These methods allow the rapid determination of the importance of individual amino acid residues in a polypeptide of interest, and can be applied to polypeptides of unknown structure.

Essential amino acids in the polypeptides of the present invention can be identified according to procedures known in the art, such as site-directed mutagenesis or alanine-scanning mutagenesis (Cunningham and Wells, Science 244:1081, 1989, Bass et al., Proc. Nat. Acad. Sci. USA 88:4498, 1991, Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699, 1996. The identities of essential amino acids can also be inferred from analysis of homologies with zacrp4.

The location of zacrp4 receptor binding domains can be identified by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306, 1992, Smith et al., J. Mol. Biol. 224:899, 1992, and Wlodaver et al., FEBS Lett 30:59, 1992. Moreover, zacrp4 labeled with biotin or FITC can be used for expression cloning of zacrp4 receptors.

The present invention also provides polypeptide fragments or peptides comprising an epitope-bearing portion of a zacrp4 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat. Acad. Sci. USA 81:3998, 1983).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660, 1983). Antibodies that recognize short, linear epitopes are particularly useful in analytic and diagnostic applications that employ denatured protein, such as Western blotting (Tobin, Proc. Natl. Acad. Sci. USA 76:4350-6, 1979), or in the analysis of fixed cells or tissue samples. Antibodies to linear epitopes are also useful for detecting fragments of zacrp4, such as might occur in body fluids or cell culture media. Accordingly, antigenic epitope-bearing peptides and polypeptides of the present invention are useful to raise antibodies that bind with the polypeptides described herein.

Antigenic epitope-bearing peptides and polypeptides preferably contain at least four to ten amino acids, at least ten to fifteen amino acids, or about 15 to about 30 amino acids of SEQ ID NO:2. Such epitope-bearing peptides and polypeptides can be produced by fragmenting a zacrp4 polypeptide, or by chemical peptide synthesis, as described herein. Moreover, epitopes can be selected by phage display of random peptide libraries (see, for example, Lane and Stephen, Curr. Opin. Immunol. 5:268, 1993, and Cortese et al., Curr. Opin. Biotechnol. 7:616, 1996). Standard methods for identifying epitopes and producing antibodies from small peptides that comprise an epitope are described, for example, by Mole, “Epitope Mapping,” in Methods in Molecular Biology, Vol. 10, Manson (ed.), pages 105-16 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

Regardless of the particular nucleotide sequence of a variant zacrp4 gene, the gene encodes a polypeptide that is characterized by its neuronal, anti-microbial, regulatory or other activities of the wild-type protein, or by the ability to bind specifically to an anti-zacrp4 antibody. More specifically, variant zacrp4 genes encode polypeptides which exhibit at least 50%, and preferably, greater than 70, 80, or 90%, of the activity of polypeptide encoded by the human zacrp4 gene described herein.

For any zacrp4 polypeptide, including variants and fusion proteins, one of ordinary skill in the art can readily generate a fully degenerate polynucleotide sequence encoding that variant using the information set forth in Tables 1 and 2 herein. Moreover, those of skill in the art can use standard software to devise zacrp4 variants based upon the nucleotide and amino acid sequences described herein. Accordingly, the present invention includes a computer-readable medium encoded with a data structure that provides at least one of the following sequences: SEQ ID NO:1, SEQ ID NO:2, and SEQ ID NO:10. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

The present invention also provides zacrp4 fusion proteins. For example, fusion proteins of the present invention encompass a polypeptide selected from the group consisting of: (a) polypeptide molecules comprising a sequence of amino acid residues as shown in SEQ ID NO:2 from amino acid residue 1 to residue 329; (b) polypeptide molecules ranging from amino acid residue 17 to residue 159 of SEQ ID NO:2, (c) polypeptide molecules ranging from amino acid residue 160 to residue 328 of SEQ ID NO:2 or (d) a portion of the zacrp4 polypeptide containing a C1q domain or an active portion of a C1q domain; and another polypeptide. The other polypeptide may be alternative or additional C1q domains, a collagen-like domain, a signal peptide to facilitate secretion of the fusion protein or the like. The globular domain of complement binds IgG, thus, the globular C1q domains of zacrp4 polypeptides, fragments or fusions may have a similar ability.

Zacrp4 polypeptides, ranging from amino acid 1 (Met) to amino acid 328 of SEQ ID NO:2; the mature zacrp4 polypeptides, ranging from amino acid 17 to amino acid 328 of SEQ ID NO:2; or the secretion leader fragments thereof, which fragments range from amino acid 1 to amino acid 16 of SEQ ID NO:2 may be used in the study of secretion of proteins from cells. In preferred embodiments of this aspect of the present invention, the mature polypeptides are formed as fusion proteins with putative secretory signal sequences; plasmids bearing regulatory regions capable of directing the expression of the fusion protein is introduced into test cells; and secretion of mature protein is monitored. The monitoring may be done by techniques known in the art, such as HPLC and the like.

The polypeptides of the present invention, including full-length proteins, fragments thereof and fusion proteins, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., ibid., and Ausubel et al. ibid.

In general, a DNA sequence encoding a zacrp4 polypeptide of the present invention is operably linked to other genetic elements required for its expression, generally including a transcription promoter and terminator within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zacrp4 polypeptide into the secretory pathway of a host cell, a secretory signal sequence (also known as a leader sequence, signal sequence, prepro sequence or pre-sequence) is provided in the expression vector. The secretory signal sequence may be that of the zacrp4 polypeptide, or may be derived from another secreted protein (e.g., t-PA) or synthesized de novo. The secretory signal sequence is joined to the zacrp4 polypeptide DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). Conversely, the signal sequence portion of the zacrp4 polypeptide (amino acids 1-16 of SEQ ID NO:2) may be employed to direct the secretion of an alternative protein by analogous methods.

The secretory signal sequence contained in the polypeptides of the present invention can be used to direct other polypeptides into the secretory pathway. The present invention provides for such fission polypeptides. A signal fusion polypeptide can be made wherein a secretory signal sequence derived from amino acid residues 1-16 of SEQ ID NO:2 is operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein, such as a receptor. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61, CHO DG44 (Chasin et al., Som. Cell. Mol. Genet. 12:555-666, 1986)) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Banalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King and Possee, The Baculovirus Expression System: A Laboratory Guide, London, Chapman & Hall; O'Reilly et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant zacrp4 baculovirus utilizes a transposon-based system described by Luckow (Luckow et al., J. Virol. 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the zacrp4 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zacrp4. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins and Possee, J. Gen. Virol. 71:971-6, 1990; Bonning et al., J. Gen. Virol. 75:1551-6, 1994; and, Chazenbalk, and Rapoport, J. Biol. Chem. 270:1543-9, 1995. In such transfer vector constructs, a short or long version of the basic protein promoter can be used. Moreover, transfer vectors can be constructed which replace the native zacrp4 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zacrp4 secretory signal sequence. In addition, transfer vectors can include an in-frame fusion with DNA encoding an epitope tag at the C- or N-terminus of the expressed zacrp4 polypeptide, for example, a Glu-Glu epitope tag (Grussenmeyer et al., Proc. Natl. Acad. Sci. 82:7952-4, 1985). Using a technique known in the art, a transfer vector containing zacrp4 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zacrp4 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells.

Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King and Possee, ibid.; O'Reilly et al., ibid.; Richardson, ibid.). Subsequent purification of the zacrp4 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075.

Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those front glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (τ) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zacrp4 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Expressed recombinant zacrp4 polypeptides (or zacrp4 polypeptide fusions) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their structural or binding properties. For example, immobilized metal ion adsorption (IMAC) chromatography can be used to purify histidine-rich proteins or proteins having a His tag. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp. 529-39). Within an additional preferred embodiments of the invention, a fusion of the polypeptide of interest and an affinity tag (e.g., maltose-binding protein, FLAG, Glu-Glu, an immunoglobulin domain) may be constructed to facilitate purification as is discussed in greater detail in the Example sections below.

Protein refolding (and optionally, reoxidation) procedures may be advantageously used. It is preferred to purify the protein to >80% purity, more preferably to >90% purity, even more preferably >95%, and particularly preferred is a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

Zacrp4 polypeptides or fragments thereof may also be prepared through chemical synthesis by methods well known in the art. Such zacrp4 polypeptides may be monomers or multimers; glycosylated or non-glycosylated; pegylated or non-pegylated; and may or may not include an initial methionine amino acid residue.

A ligand-binding polypeptide, such as a zacrp4-binding polypeptide, can also be used for purification of ligand. The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the ligand-binding polypeptide. The ligand is then eluted using changes in salt concentration, chaotropic agents (guanidine HCl), or pH to disrupt ligand-receptor binding.

Nucleic acid molecules disclosed herein can be used to detect the expression of a zacrp4 gene in a biological sample. Such probe molecules include double-stranded nucleic acid molecules comprising the nucleotide sequences of SEQ ID NO: 1, or fragments thereof, as well as single-stranded nucleic acid molecules having the complement of the nucleotide sequences of SEQ ID NO:1, or a fragment thereof. Probe molecules may be DNA, RNA, oligonucleotides, and the like.

As an illustration, suitable probes include nucleic acid molecules that bind with a portion of a zacrp4 domain or motif, such as the nucleotide sequence that encode the amino-terminal signal sequence (residues 1-16 of SEQ ID NO:2), the two tandem C1q domains (residues 17-159 and 160-328 of SEQ ID NO:2), aromatic motif (F-X(5)-[ND]-X(4)-[FYWL]-X(6)-F-X(5)-G-X-Y-X-F-X-[FY] (SEQ ID NO:3), the 10 beta-strands amino acid residues 31-35, 52-54, 60-63, 67-69, 73-84, 89-95, 101-108, 112-126, 131-136 and 150-154, and 179-183, 206-208, 214-217, 221-223, 227-238, 243-249, 254-262, 267-278, 283-288 and 305-309 of SEQ ID NO:2, receptor binding loops 37-62, 94-108, 185-216 and 248-262 of SEQ ID NO:2.

In a basic assay, a single-stranded probe molecule is incubated with RNA, isolated from a biological sample, under conditions of temperature and ionic strength that promote base pairing between the probe and target zacrp4 RNA species, as described herein. After separating unbound probe from hybridized molecules, the amount of hybrids is detected.

Well-established hybridization methods of RNA detection include northern analysis and dot/slot blot hybridization (see, for example, Ausubel ibid. and Wu et al. (eds.), “Analysis of Gene Expression at the RNA Level,” in Methods in Gene Biotechnology, pages 225-239 (CRC Press, Inc. 1997)). Nucleic acid probes can be detectably labeled with radioisotopes such as ³²P or ³⁵S. Alternatively, zacrp4 RNA can be detected with a nonradioactive hybridization method (see, for example, Isaac (ed.), Protocols for Nucleic Acid Analysis by Nonradioactive Probes, Humana Press, Inc., 1993). Typically, nonradioactive detection is achieved by enzymatic conversion of chromogenic or chemiluminescent substrates. Illustrative nonradioactive moieties include biotin, fluorescein, and digoxigenin.

Zacrp4 oligonucleotide probes are also useful for in vivo diagnosis. As an illustration, ⁸F-labeled oligonucleotides can be administered to a subject and visualized by positron emission tomography (Tavitian et al., Nature Medicine 4:467, 1998).

Numerous diagnostic procedures take advantage of the polymerase chain reaction (PCR) to increase sensitivity of detection methods. Standard techniques for performing PCR are well-known (see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)). PCR primers can be designed to amplify a sequence encoding a particular zacrp4 domain or motif, as described above.

One variation of PCR for diagnostic assays is reverse transcriptase-PCR (RT-PCR). In the RT-PCR technique, RNA is isolated from a biological sample, reverse transcribed to cDNA, and the cDNA is incubated with zacrp4 primers (see, for example, Wu et al. (eds.), “Rapid Isolation of Specific cDNAs or Genes by PCR,” in Methods in Gene Biotechnology, CRC Press, Inc., pages 15-28, 1997). PCR is then performed and the products are analyzed using standard techniques.

As an illustration, RNA is isolated from biological sample using, for example, the guanidinium-thiocyanate cell lysis procedure described above. Alternatively, a solid-phase technique can be used to isolate mRNA from a cell lysate. A reverse transcription reaction can be primed with the isolated RNA using random oligonucleotides, short homopolymers of dT, or zacrp4 anti-sense oligomers. Oligo-dT primers offer the advantage that various mRNA nucleotide sequences are amplified that can provide control target sequences. Zacrp4 sequences are amplified by the polymerase chain reaction using two flanking oligonucleotide primers that are typically at least 5 bases in length.

PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled zacrp4 probe, and examined by autoradiography. Additional alternative approaches include the use of digoxigenin-labeled deoxyribonucleic acid triphosphates to provide chemiluminescence detection, and the C-TRAK calorimetric assay.

Another approach is real time quantitative PCR (Perkin-Elmer Cetus, Norwalk, Ct.). A fluorogenic probe, consisting of an oligonucleotide with both a reporter and a quencher dye attached, anneals specifically between the forward and reverse primers. Using the 5′ endonuclease activity of Taq DNA polymerase, the reporter dye is separated from the quencher dye and a sequence-specific signal is generated and increases as amplification increases. The fluorescence intensity can be continuously monitored and quantified during the PCR reaction.

Another approach for detection of zacrp4 expression is cycling probe technology (CPT), in which a single-stranded DNA target binds with an excess of DNA-RNA-DNA chimeric probe to form a complex, the RNA portion is cleaved with RNase H, and the presence of cleaved chimeric probe is detected (see, for example, Beggs et al., J. Clin. Microbiol. 34:2985, 1996 and Bekkaoui et al., Biotechniques 20:240, 1996). Alternative methods for detection of zacrp4 sequences can utilize approaches such as nucleic acid sequence-based amplification (NASBA), cooperative amplification of templates by cross-hybridization (CATCH), and the ligase chain reaction (LCR) (see, for example, Marshall et al., U.S. Pat. No. 5,686,272 (1997), Dyer et al., J. Virol. Methods 60:161, 1996; Ehricht et al., Eur. J. Biochem. 243:358, 1997 and Chadwick et al., J. Virol. Methods 70:59, 1998). Other standard methods are known to those of skill in the art.

Zacrp4 probes and primers can also be used to detect and to localize zacrp4 gene expression in tissue samples. Methods for such in situ hybridization are well-known to those of skill in the art (see, for example, Choo (ed.), In Situ Hybridization Protocols, Humana Press, Inc., 1994; Wu et al. (eds.), “Analysis of Cellular DNA or Abundance of mRNA by Radioactive In Situ Hybridization (RISH),” in Methods in Gene Biotechnology, CRC Press, Inc., pages 259-278, 1997 and Wu et al. (eds.), “Localization of DNA or Abundance of mRNA by Fluorescence In Situ Hybridization (RISH),” in Methods in Gene Biotechnology, CRC Press, Inc., pages 279-289, 1997).

Various additional diagnostic approaches are well-known to those of skill in the art (see, for example, Mathew (ed.), Protocols in Human Molecular Genetics Humana Press, Inc., 1991; Coleman and Tsongalis, Molecular Diagnostics, Humana Press, Inc., 1996 and Elles, Molecular Diagnosis of Genetic Diseases, Humana Press, Inc., 1996).

An assay system that uses a ligand-binding receptor (or an antibody, one member of a complement/anti-complement pair) or a binding fragment thereof, and a commercially available biosensor instrument (BIAcore™, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

The invention also provides anti-zacrp4 antibodies. Antibodies to zacrp4 can be obtained, for example, using as an antigen the product of a zacrp4 expression vector, or zacrp4 isolated from a natural source. Particularly useful anti-zacrp4 antibodies “bind specifically” with zacrp4. Antibodies are considered to be specifically binding if the antibodies bind to a zacrp4 polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660, 1949). Suitable antibodies include antibodies that bind with zacrp4 in particular domains.

Anti-zacrp4 antibodies can be produced using antigenic zacrp4 epitope-bearing peptides and polypeptides. Antigenic epitope-bearing peptides and polypeptides of the present invention contain a sequence of at least nine, preferably between 15 to about 30 amino acids contained within SEQ ID NO:2. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that bind with zacrp4. It is desirable that the amino acid sequence of the epitope-bearing peptide is selected to provide substantial solubility in aqueous solvents (i.e., the sequence includes relatively hydrophilic residues, while hydrophobic residues are preferably avoided). Hydrophilic peptides can be predicted by one of skill in the art from a hydrophobicity plot, see for example, Hopp and Woods (Proc. Nat. Acad. Sci. USA 78:3824-8, 1981) and Kyte and Doolittle (J. Mol. Biol. 157: 105-142, 1982). Moreover, amino acid sequences containing proline residues may be also be desirable for antibody production.

Polyclonal antibodies to recombinant zacrp4 protein or to zacrp4 isolated from natural sources can be prepared using methods well-known to those of skill in the art. See, for example, Green et al., “Production of Polyclonal Antisera,” in Immunochemical Protocols (Manson, ed.), pages 1-5 (Humana Press 1992), and Williams et al., “Expression of foreign proteins in E. Coli using plasmid vectors and purification of specific polyclonal antibodies,” in DNA Cloning 2: Expression Systems, 2nd Edition, Glover et al. (eds.), page 15 (Oxford University Press 1995). The immunogenicity of a zacrp4 polypeptide can be increased through the use of an adjuvant, such as alum (aluminum hydroxide) or Freund's complete or incomplete adjuvant. Polypeptides useful for immunization also include fusion polypeptides, such as fusions of zacrp4 or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, hamsters, guinea pigs, goats, or sheep, an anti-zacrp4 antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310, 1990. Antibodies can also be raised in transgenic animals such as transgenic sheep, cows, goats or pigs, and can also be expressed in yeast and fungi in modified forms as will as in mammalian and insect cells.

Alternatively, monoclonal anti-zacrp4 antibodies can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.12.6.7 (John Wiley & Sons 1991), Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a zacrp4 gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an anti-zacrp4 antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13, 1994, Lonberg et al., Nature 368:856, 1994, and Taylor et al., Int. Immun. 6:579, 1994.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-zacrp4 antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230, 1960, Porter, Biochem. J. 73:119, 1959, Edelman et al., in Methods in Enzymology Vol. 1, page 422 (Academic Press 1967), and by Coligan, ibid.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Natl. Acad. Sci. USA 69:2659, 1972. Alternatively, the variable chains can be linked by an intermolecular disulfide bond or cross-linked by chemicals such as gluteraldehyde (see, for example, Sandhu, Crit. Rev. Biotech 12:437, 1992).

The Fv fragments may comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97, 1991, also see, Bird et al., Science 242:423, 1988, Ladner et al., U.S. Pat. No. 4,946,778, Pack et al., Bio/Technoloy 11:1271, 1993, and Sandhu, ibid.

As an illustration, a scFV can be obtained by exposing lymphocytes to zacrp4 polypeptide in vitro, and selecting antibody display libraries in phage or similar vectors (for instance, through use of immobilized or labeled zacrp4 protein or peptide). Genes encoding polypeptides having potential zacrp4 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the zacrp4 sequences disclosed herein to identify proteins which bind to zacrp4.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106, 1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press, 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-zacrp4 antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat. Acad. Sci. USA 86:3833, 1989. Techniques for producing humanized monoclonal antibodies are described, for example, by Jones et al., Nature 321:522, 1986, Carter et al., Proc. Nat. Acad. Sci. USA 89:4285, 1992, Sandhu, Crit. Rev. Biotech 12:437, 1992, Singer et al., J. Immun. 150:2844, 1993, Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399-434 (John Wiley & Sons, Inc. 1996), and by Queen et al., U.S. Pat. No. 5,693,762 (1997).

Polyclonal anti-idiotype antibodies can be prepared by immunizing animals with anti-zacrp4 antibodies or antibody fragments, using standard techniques. See, for example, Green et al., “Production of Polyclonal Antisera,” in Methods In Molecular Biology: Immunochemical Protocols, Manson (ed.), pages 1-12 (Humana Press 1992). Also, see Coligan, ibid. at pages 2.4.1-2.4.7. Alternatively, monoclonal anti-idiotype antibodies can be prepared using anti-zacrp4 antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J.

Gen. Virol. 77:1875, 1996. Genes encoding polypeptides having potential zacrp4 polypeptide binding domains, “binding proteins”, can be obtained by screening random or directed peptide libraries displayed on phage (phage display) or on bacteria, such as E. coli. Nucleotide sequences encoding the polypeptides can be obtained in a number of ways, such as through random mutagenesis and random polynucleotide synthesis. Alternatively, constrained phage display libraries can also be produced. These peptide display libraries can be used to screen for peptides which interact with a known target which can be a protein or polypeptide, such as a ligand or receptor, a biological or synthetic macromolecule, or organic or inorganic substances. Techniques for creating and screening such peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Peptide display libraries can be screened using the zacrp4 sequences disclosed herein to identify proteins which bind to zacrp4. These “binding proteins” which interact with zacrp4 polypeptides can be used essentially like an antibody.

A variety of assays known to those skilled in the art can be utilized to detect antibodies and/or binding proteins which specifically bind to zacrp4 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zacrp4 protein or polypeptide.

Antibodies and binding proteins to zacrp4 may be used for tagging cells that express zacrp4; for isolating zacrp4 by affinity purification; for diagnostic assays for determining circulating levels of zacrp4 polypeptides; for detecting or quantitating soluble zacrp4 as marker of underlying pathology or disease; in analytical methods employing FACS; for screening expression libraries; for generating anti-idiotypic antibodies; and as neutralizing antibodies or as antagonists to block zacrp4 polypeptide modulation of spermatogenesis or like activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Moreover, antibodies to zacrp4 or fragments thereof may be used in vitro to detect denatured zacrp4 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zacrp4 polypeptides or anti-zacrp4 antibodies, or bioactive fragments or portions thereof, can be coupled to detectable or cytotoxic molecules and delivered to a mammal having cells, tissues or organs that express the anti-complementary molecule.

An additional aspect of the present invention provides methods for identifying agonists or antagonists of the zacrp4 polypeptides disclosed above, which agonists or antagonists may have valuable properties as discussed further herein. Within one embodiment, there is provided a method of identifying zacrp4 polypeptide agonists, comprising providing cells responsive thereto, culturing the cells in the presence of a test compound and comparing the cellular response with the cell cultured in the presence of the zacrp4 polypeptide, and selecting the test compounds for which the cellular response is of the same type.

Within another embodiment, there is provided a method of identifying antagonists of zacrp4 polypeptide, comprising providing cells responsive to a zacrp4 polypeptide, culturing a first portion of the cells in the presence of zacrp4 polypeptide, culturing a second portion of the cells in the presence of the zacrp4 polypeptide and a test compound, and detecting a decrease in a cellular response of the second portion of the cells as compared to the first portion of the cells. In addition to those assays disclosed herein, samples can be tested for inhibition of zacrp4 activity within a variety of assays designed to measure receptor binding or the stimulation/inhibition of zacrp4-dependent cellular responses. For example, zacrp4-responsive cell lines can be transfected with a reporter gene construct that is responsive to a zacrp4-stimulated cellular pathway. Reporter gene constructs of this type are known in the art, and will generally comprise a zacrp4-DNA response element operably linked to a gene encoding an assayable protein, such as luciferase. DNA response elements can include, but are not limited to, cyclic AMP response elements (CRE), hormone response elements (HRE), insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6, 1988 and Habener, Molec. Endocrinol. 4 (8):1087-94, 1990. Hormone response elements are reviewed in Beato, Cell 56:335-44; 1989. Candidate compounds, solutions, mixtures or extracts are tested for the ability to inhibit the activity of zacrp4 on the target cells as evidenced by a decrease in zacrp4 stimulation of reporter gene expression. Assays of this type will detect compounds that directly block zacrp4 binding to cell-surface receptors, as well as compounds that block processes in the cellular pathway subsequent to receptor-ligand binding. In the alternative, compounds or other samples can be tested for direct blocking of zacrp4 binding to receptor using zacrp4 tagged with a detectable label (e.g., ¹⁰²⁵I, biotin, horseradish peroxidase, FITC, and the like). Within assays of this type, the ability of a test sample to inhibit the binding of labeled zacrp4 to the receptor is indicative of inhibitory activity, which can be confirmed through secondary assays. Receptors used within binding assays may be cellular receptors or isolated, immobilized receptors.

Zacrp4 is a secreted protein which functions as a primary message involved in cell to cell communication. As such zacrp4 polypeptides, fragments and fusions may act as paracrine factors, exerting their influence on distant targets. Zacrp4 polypeptides, fragments and fusions may be useful as autocrine factors, particularly during development. Acting as endocrine factors, zacrp4 polypeptides, fragments and fusions are useful in mediating the processes of an organism. Zacrp4 polypeptides, fragments and fusions would be useful in regulating cellular processes such as cell proliferation and/or differentiation, cell survival and energy balance.

Zacrp4 is highly expressed in testis and ovarian tissues. Testis-specific factors that influence spermatogenesis may come directly from the Sertoli cells that are in contact with spermatogenic cells, or may be paracrine or endocrine factors. Paracrine factors that cross the cellular barrier and enter the sperm cell microenvironment include molecules secreted from Leydig cells. Leydig cells produce several factors believed to play an important role in spermatogenic cell-maturation process, such as testosterone, Leydig factor, IGF-1, inhibin and activin. Zacrp4 expression in the testis suggests a role in the spermatogenic cycle. As such, zacrp4 polypeptides, fragments; fusions and the like may serve in sperm cell maturation, testis cell-cell interactions, regulating spermatogenesis by affecting proliferation or differentiation of spermatogenic cells, affecting cell-cell interactions, modulating hormones involved in the process, sperm motility, capacitation, zona interaction and the like.

In vivo assays for evaluating the effect of polypeptides, such as zacrp4, on testes are well known in the art. For example, compounds can be injected intraperitoneally into animals for a specific time duration. After the treatment period, the animals are sacrificed and testes removed and weighed. Testicles are homogenized and sperm head counts are made (Meistrich et al., Exp. Cell Res. 99:72-78, 1976).

Other activities, such as chemotaxic activity that may be associated with proteins of the present invention can be analyzed using methods known in the art. For example, late stage factors in spermatogenesis may be involved in egg-sperm interactions and sperm motility. Activities, such as enhancing viability of cryopreserved sperm, stimulating the acrosome reaction, enhancing sperm motility and enhancing egg-sperm interactions may be associated with the proteins of the present invention. Assays evaluating such activities can be found, for example, in Rosenberger, J. Androl. 11:89-96, 1990; Fuchs, Zentralbl Gynakol 11:117-120, 1993; Neurwinger et al., Andrologia 22:335-9, 1990; Harris et al., Human Reprod. 3:856-60, 1988; Jockenhovel, Andrologia 22:171-178, 1990; Lessing et al., Fertil. Steril. 44:406-9 (1985) and Zaneveld, In Male Infertility Chapter 11, Comhaire Ed., Chapman & Hall, London 1996. These activities are expected to result in enhanced fertility and successful reproduction.

Zacrp4 expression in ovaries suggests a role in oogenesis. The classic control of ovarian function by luteinizing hormone (LH) and follicle stimulating hormone (FSH) is now thought to include the action of a variety of other molecules that act to promote cell-cell interactions between cells of the follicle, for review, see Gougeon, Endocrine Rev. 17:121-55, 1996. As such, zacrp4 polypeptides, fragments, fusions and the like may serve in oocyte maturation, ovarian cell-cell interactions, regulating folliculogenesis and dominant follicle selection, by affecting proliferation or differentiation of follicular cells, affecting cell-cell interactions, modulating hormones involved in the process, promoting uterine implantation of fertilized oocytes and the like.

Zacrp4 is also expressed at high levels in brain tissue. Regulation of reproductive function in males and females is controlled, in part, by feedbaek inhibition of the hypothalamus and anterior pituitary by blood-bone hormones. Gonadotropin-releasing hormone is secreted from the hypothalamus and controls the reproductive hormone cascade directing synthesis and release of pituitary gonadotropins through specific high-affinity membrane bound receptors. These gonadotropins, luteinizing hormone (LH) and follicle stimulating hormone (FSH), in turn regulate the activity of testes and ovaries. Modulators of their activity is desirable for therapeutic applications such as precocious puberty, endometriosis, uterine leiomyomata, hirsutism, infertility, pre-menstrual syndrome, amenorrhea and as contraceptive agents. Such applications would also include breakthrough menopausal bleeding, as part of a therapeutic regime for pregnancy support, or for treating symptoms associated with polycystic ovarian syndrome (PCOS), PMS and menopause. Testis proteins, such as activins and inhibins, have been shown to regulate secretion of active molecules including FSH for the pituitary (Ying, Endodcr. Rev. 9:267-93, 1988; Plant et al., Hum. Reprod. 8:41-44, 1993). Inhibins and relaxin are also expressed in the ovaries have also been shown to regulate ovarian functions and follicular and uterine growth (Woodruff et al., Endocr. 132:2332-42, 1993; Russell et al., J. Reprod. Fertil. 100:115-22, 1994; Bagnell et al., J. Reprod. Fertil. 48:127-38, 1993). Zacrp4 polypeptide, fragments and fusions may act to modulate the hormones associated with the reproductive cascade.

In addition, the present invention also provides methods for studying steroidogenesis and steroid hormone secretion. For example, such a method would comprise incubating ovarian cells in culture medium comprising zacrp4 polypeptides, fragments, fusions, monoclonal antibodies, agonists or antagonists thereof with and without gonadotropins and/or steroid hormones, and subsequently observing protein and steroid secretion. Exemplary gonadotropin hormones include luteinizing hormone and follicle stimulating hormone (Rouillier et al., Mol. Reprod. Dev. 50:170-7, 1998). Exemplary steroid hormones include estradiol, androstenedione, and progesterone. Effects of zacrp4 on steroidogenesis or steroid secretion can be determined by methods known in the art, such as radioimmunoassay (to detect levels of estradiol, androstenedione, progesterone, and the like), and immunoradiometric assay (IRMA).

Molecules expressed in the ovaries and testis, such as zacrp4, which may modulate hormones, hormone receptors, growth factors, or cell-cell interactions, of the reproductive cascade or are involved in oocyte or ovarian development, sperm or testis development, would be useful as markers for cancer of reproductive organs and as therapeutic agents for hormone-dependent cancers, by inhibiting hormone-dependent growth and/or development of tumor cells. Moreover, receptors for steroid hormones involved in the reproductive cascade are found in human tumors and tumor cell lines (breast, prostate, endometrial, ovarian, kidney, and pancreatic tumors) (Kakar et al., Mol. Cell. Endocrinol., 106:145-49, 1994; Kakar and Jennes, Cancer Letts., 98:57-62, 1995). Thus, expression of zacrp4 in ovaries and testis suggests that polypeptides of the present invention would be useful in diagnostic methods for the detection and monitoring of reproductive cell cancers.

Zacrp4 polypeptides, fragments, fusions, agonists or antagonists can be used to modulate energy balance in mammals. In this regard zacrp4 polypeptides would modulate cellular metabolic reactions. Such metabolic reactions include adipogenesis, gluconeogenesis, glycogenolysis, lipogenesis, glucose uptake, protein synthesis, thermogenesis, oxygen utilization and the like.

Among other methods known in the art or described herein, mammalian energy balance may be evaluated by monitoring one or more of the following metabolic functions: adipogenesis, gluconeogenesis, glycogenolysis, lipogenesis, glucose uptake, protein synthesis, thermogenesis, oxygen utilization or the like. These metabolic functions are monitored by techniques (assays or animal models) known to one of ordinary skill in the art, as is more fully set forth below. For example, the glucoregulatory effects of insulin are predominantly exerted in the liver, skeletal muscle and adipose tissue. Insulin binds to its cellular receptor in these three tissues and initiates tissue-specific actions that result in, for example, the inhibition of glucose production and the stimulation of glucose utilization. In the liver, insulin stimulates glucose uptake and inhibits gluconeogenesis and glycogenolysis. In skeletal muscle and adipose tissue, insulin acts to stimulate the uptake, storage and utilization of glucose.

Art-recognized methods exist for monitoring all of the metabolic functions recited above. Thus, one of ordinary skill in the art is able to evaluate zacrp4 polypeptides, fragments, fusion proteins, antibodies, agonists and antagonists for metabolic modulating functions. Exemplary modulating techniques are set forth below.

Adipogenesis, gluconeogenesis and glycogenolysis are interrelated components of mammalian energy balance, which may be evaluated by known techniques using, for example, ob/ob mice or db/db mice. The ob/ob mice are inbred mice that are homozygous for an inactivating mutation at the ob (obese) locus. Such ob/ob mice are hyperphagic and hypometabolic, and are believed to be deficient in production of circulating OB protein. The db/db mice are inbred mice that are homozygous for an inactivating mutation at the db (diabetes) locus. The db/db mice display a phenotype similar to that of ob/ob mice, except db/db mice also display a diabetic phenotype. Such db/db mice are believed to be resistant to the effects of circulating OB protein. Also, various in vitro methods of assessing these parameters are known in the art.

Insulin-stimulated lipogenesis, for example, may be monitored by measuring the incorporation of ¹⁴C-acetate into triglyceride (Mackall et al. J. Biol. Chem. 251:64624, 1976) or triglyceride accumulation (Kletzien et al., Mol. Pharmacol. 41:393-8, 1992).

Glucose uptake may be evaluated, for example, in an assay for insulin-stimulated glucose transport. Non-transfected, differentiated L6 myotubes (maintained in the absence of G418) are placed in DMEM containing 1 g/l glucose, 0.5 or 1.0% BSA, 20 mM Hepes, and 2 mM glutamine. After two to five hours of culture, the medium is replaced with fresh, glucose-free DMEM containing 0.5 or 1.0% BSA, 20 mM Hepes, 1 mM pyruvate, and 2 mM glutamine. Appropriate concentrations of insulin or IGF-l, or a dilution series of the test substance, are added, and the cells are incubated for 20-30 minutes. ³H or ¹⁴C-labeled deoxyglucose is added to ≈50 ┐M final concentration, and the cells are incubated for approximately 10-30 minutes. The cells are then quickly rinsed with cold buffer (e.g. PBS), then lysed with a suitable lysing agent (e.g. 1% SDS or 1 N NaOH). The cell lysate is then evaluated by counting in a scintillation counter. Cell-associated radioactivity is taken as a measure of glucose transport after subtracting non-specific binding as determined by incubating cells in the presence of cytocholasin b, an inhibitor of glucose transport. Other methods include those described by, for example, Manchester et al., Am. J. Physiol. 266 (Endocrinol. Metab. 29):E326-E333, 1994 (insulin-stimulated glucose transport).

Protein synthesis may be evaluated, for example, by comparing precipitation of ³⁵S-methionine-labeled proteins following incubation of the test cells with ³⁵S-methionine and ³⁵S-methionine and a putative modulator of protein synthesis.

Thermogenesis may be evaluated as described by B. Stanley in The Biology of Neuropeptide Y and Related Peptides, Colmers and Wahlestedt (eds.), Humana Press, Ottawa, 1993, pp. 457-509; Billington et al., Am. J. Physiol. 260:R321, 1991; Zarjevski et al., Endocrinology 133:1753, 1993; Billington et al., Am. J. Physiol. 266:R1765, 1994; Heller et al., Am. J. Physiol. 252(4 Pt 2): R661-7, 1987; and Heller et al., Am. J. Physiol. 245: R321-8, 1983. Also, metabolic rate, which may be measured by a variety of techniques, is an indirect measurement of thermogenesis.

Oxygen utilization may be evaluated as described by Heller et al., Pflugers Arch 369: 55-9, 1977. This method also involved an analysis of hypothalmic temperature and metabolic heat production. Oxygen utilization and thermoregulation have also been evaluated in humans as described by Haskell et al., J. Appl. Physiol. 51: 948-54, 1981.

Zacrp4 polypeptides may be used in the analysis of energy efficiency of a mammal. Zacrp4 polypeptides found in serum or tissue samples may be indicative of a mammals ability to store food, with more highly efficient mammals tending toward obesity. More specifically, the present invention contemplates methods for detecting zacrp4 polypeptide comprising:

exposing a sample possibly containing zacrp4 polypeptide to an antibody attached to a solid support, wherein said antibody binds to an epitope of a zacrp4 polypeptide;

washing said immobilized antibody-polypeptide to remove unbound contaminants;

exposing the immobilized antibody-polypeptide to a second antibody directed to a second epitope of a zacrp4 polypeptide, wherein the second antibody is associated with a detectable label; and

detecting the detectable label. The concentration of zacrp4 polypeptide in the test sample appears to be indicative of the energy efficiency of a mammal. This information can aid nutritional analysis of a mammal. Potentially, this information may be useful in identifying and/or targeting energy deficient tissue.

A further aspect of the invention provides a method for studying insulin. Such methods of the present invention comprise incubating adipocytes in a culture medium comprising zacrp4 polypeptide, monoclonal antibody, agonist or antagonist thereof ±insulin and observing changes in adipocyte protein secretion or differentiation.

The present invention also provides methods of studying mammalian cellular metabolism. Such methods of the present invention comprise incubating cells to be studied, for example, human vascular endothelial cells, ±zacrp4 polypeptide, monoclonal antibody, agonist or antagonist thereof and observing changes in adipogenesis, gluconeogenesis, glycogenolysis, lipogenesis, glucose uptake, or the like.

Expression in the brain and spinal cord suggests that zacrp4 polypeptides, fragments, fusions and the like may be involved in neuronal signal propagation or guidance of neuronal cell migration. Cerebellin, a member of the C1q family is involved in synaptic activity (Urade et al., Proc. Natl. Acad. Sci. USA 88:1069-73, 1991). As neurotransmitters or neurotransmission modulators, zacrp4 polypeptide fragments as well as zacrp4 polypeptides, fusion proteins, agonists, antagonists or antibodies of the present invention may also modulate calcium ion concentration, muscle contraction, hormone secretion, DNA synthesis or cell growth, inositol phosphate turnover, arachidonate release, phospholipase-C activation, gastric emptying, human neutrophil activation or ADCC capability, superoxide anion production and the like. Evaluation of these properties can be conducted by known methods, such as those set forth herein.

Among other methods known in the art or described herein, neurotransmission functions may be evaluated by monitoring 2-deoxy-glucose uptake in the brain. This parameter is monitored by techniques (assays or animal models) known to one of ordinary skill in the art, for example, autoradiography. Useful monitoring techniques are described, for example, by Kilduff et al., J. Neurosci. 10 2463-75, 1990, with related techniques used to evaluate the “hibernating heart” as described in Gerber et al. Circulation 94: 651-8, 1996, and Fallavollita et al., Circulation 95: 1900-9, 1997.

Neurite growth cues are of great therapeutic value. Proteins involved in the guidance of neural cell migration would be of value for example, in modulating neurite growth and development; treatment of peripheral neuropathies; for use as therapeutics for the regeneration of neurons following strokes, brain damage caused by head injuries and paralysis caused by spinal injuries; diagnosing neurological diseases and in treating neurodegenerative diseases such as multiple sclerosis, Alzheimer's disease and Parkinson's disease.

The ability of zacrp4 polypeptides, fragments, fusions, agonists, antagonists and antibodies of the present invention to guide neural cell migration can be measured using assays known in the art. For example, changes in neuron growth patterns in response to exogenous proteins, can be measured according to the procedures disclosed in Hastings, WIPO Patent Application No:97/29189 and Walter et al., Development 101:685-96, 1987. Other assays such as the C assay (see for example, Raper and Kapfhammer, Neuron 4:21-9, 1990 and Luo et al., Cell 75:217-27, 1993), can be used to determine whether an exogenously added protein induces collapsing activity on growing neurons. Other methods which assess inhibition of neurite extension or divert such extension are also known, see Goodman, Annu. Rev. Neurosci. 19:341-77, 1996. Conditioned media from cells expressing the desired protein, protein agonists or antagonists, or aggregates of such protein expressing cells, can by placed in a gel matrix near suitable neural cells, such as dorsal root ganglia (DRG) or sympathetic ganglia explants, which have been cocultured with nerve growth factor. Compared to control cells, exogenous protein-induced changes in neuron growth can be measured (see for example, Messersmith et al., Neuron 14:949-59, 1995; Puschel et al., Neuron 14:941-8, 1995). Likewise neurite outgrowth can be measured using neuronal cell suspensions grown in the presence of molecules of the present invention see for example, O'Shea et al., Neuron 7:231-7, 1991 and DeFreitas et al., Neuron 15:333-43, 1995.

Complement component C1q plays a role in host defense against infectious agents, such as bacteria and viruses. C1q is known to exhibit several specialized functions. For example, C1q triggers the complement cascade via interaction with bound antibody or C-reactive protein (CRP). Also, C1q interacts directly with certain bacteria, RNA viruses, mycoplasma, uric acid crystals, the lipid A component of bacterial endotoxin and membranes of certain intracellular organelles. C1q binding to the C1q receptor is believed to promote phagocytosis. C1q also appears to enhance the antibody formation aspect of the host defense system. See, for example, Johnston, Pediatr. Infect. Dis. J. 12: 93341, 1993. Thus, soluble C1q-like molecules may be useful as anti-microbial agents, promoting lysis or phagocytosis of infectious agents.

Zacrp4 fragments as well as zacrp4 polypeptides, fusion proteins, agonists, antagonists or antibodies may be evaluated with respect to their anti-microbial properties according to procedures known in the art. See, for example, Barsum et al., Eur. Respir. J. 8(5): 709-14; 1995; Sandovsky-Losica et al., J. Med. Vet. Mycol (England) 28(4): 279-87, 1990; Mehentee et al., J. Gen. Microbiol. (England) 135 (Pt. 8): 2181-8, 1989; Segal and Savage, J. Med. Vet. Mycol. 24: 477-9, 1986 and the like. If desired, the performance of zacrp4 in this regard can be compared to proteins known to be functional in this regard, such as proline-rich proteins, lysozyme, histatins, lactoperoxidase or the like. In addition, zacrp4 fragments, polypeptides, fusion proteins, agonists, antagonists or antibodies may be evaluated in combination with one or more anti-microbial agents to identify synergistic effects. One of ordinary skill in the art will recognize that the anti-microbial properties of zacrp4 polypeptides, fragments, fusion proteins, agonists, antagonists and antibodies may be similarly evaluated.

Anti-microbial protective agents may be directly acting or indirectly acting. Such agents operating via membrane association or pore forming mechanisms of action directly attach to the offending microbe. Anti-microbial agents can also act via an enzymatic mechanism, breaking down microbial protective substances or the cell wall/membrane thereof. Anti-microbial agents, capable of inhibiting microorganism proliferation or action or of disrupting microorganism integrity by either mechanism set forth above, are useful in methods for preventing contamination in cell culture by microbes susceptible to that anti-microbial activity. Such techniques involve culturing cells in the presence of an effective amount of said zacrp4 polypeptide or an agonist or antagonist thereof.

Also, zacrp4 polypeptides or agonists thereof may be used as cell culture reagents in in vitro studies of exogenous microorganism infection, such as bacterial, viral or fungal infection. Such moieties may also be used in in vivo animal models of infection.

In view of the tissue distribution observed for zacrp4, agonists (including the natural ligand/substrate/cofactor/etc.) and antagonists have enormous potential in both in vitro and in vivo applications. Compounds identified as zacrp4 agonists are useful for stimulating cell growth or development in vitro and in vivo. For example, zacrp4 and agonist compounds are useful as components of defined cell culture media, and may be used alone or in combination with other cytokines and hormones to replace serum that is commonly used in cell culture. Agonists are thus useful in specifically promoting the growth and/or development of cells in culture.

For example, considering the high expression of zacrp4 in testis and ovary, zacrp4 polypeptides, fragments, fusions and agonists may be particularly useful as research reagents to stimulate proliferation, maturation and differentiation of testicular cells, ovarian cells, eggs, sperm, cells from animal embryos or primary cultures derived from these tissues. Proliferation and differentiation can be measured using cultured cells or by in vivo administration of molecules of the present invention to the appropriate animal model. Cultured testicular cells include dolphin DB1.Tes cells (CRL-6258); mouse GC-1 spg cells (CRL-2053); TM3 cells (CRL-1714); TM4 cells (CRL-1715); and pig ST cells (CRL-1746), available from American Type Culture Collection, 12301 Parklawn Drive, Rockville, Md.

Assays measuring cell proliferation or differentiation are well known in the art. Such methods include incubating granulosa cells, theca cells, oocytes or a combination thereof, in the presence and absence of a zacrp4 polypeptide, fragment, fusion, monoclonal antibody, agonist or antagonist thereof and observing changes in cell proliferation, maturation and differentiation. See for example, Basini et al.(J. Rep. Immunol. 37:139-53, 1998); Duleba et al.,(Fert. Ster. 69:335-40, 1998); and Campbell et al., J. Reprod. and Fert. 112:69-77, 1998).

Other assays measuring proliferation include such assays as chemosensitivity to neutral red dye (Cavanaugh et al., Investigational New Drugs 8:347-354, 1990), incorporation of radiolabelled nucleotides (Cook et al., Analytical Biochem. 179:1-7, 1989), incorporation of 5-bromo-2′-deoxyuridine (BrdU) in the DNA of proliferating cells (Porstmann et al., J. Immunol. Methods 82:169-179, 1985), and use of tetrazolium salts (Mosmann, J. Immunol. Methods 65:55-63, 1983; Alley et al., Cancer Res. 48:589-601, 1988; Marshall et al., Growth Reg. 5:69-84, 1995; and Scudiero et al., Cancer Res. 48:48274833, 1988). Another dye incorporation assay to quantitatively measure the proliferation of cells uses alamar Blue™ (AccuMed, Chicago, Ill.) to indicate a colorimetric change which is quantitated by fluorescent signal (Lancaster et al., U.S. Pat. No. 5,501,959).

Assays measuring differentiation include, for example, measuring cell-surface markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989).

An additional aspect of the invention provides a method for studying trimerization. Such methods of the present invention comprise incubating zacrp4 polypeptides or fragments or fusion proteins thereof containing one or both C1q domains alone or in combination with other polypeptides bearing C1q domains. Such associations may compete with the formation of complement C1q trimers and thereby disrupt serum complement activity. The impact of zacrp4 polypeptides, fragments, fusions, agonists or antagonists on complement inhibition may be assessed by methods known in the art.

Adipocyte complement related proteins are involved in cell-cell or cell-extracellular matrix interactions, particularly those involving modulation of tissue remodeling. The phenotypic manifestation of many autoimmune and remodeling-related diseases is extensive activation of inflammatory and/or tissue remodeling processes. The result is often that functional organ or sub-organ tissue is replaced by a variety of extracellular matrix (ECM) components incapable of performing the function of the replaced biological structure. There is an incomplete understanding of the initiation events in these diseases, and the resulting excessive extracellular matrix deposition. The initiation events have been hypothesized to involve an injury or initial perturbation of the optimal biological structure regulation. Interestingly, sometimes intracellular components are found as autoantigens, indicative of particular diseases. It could be that the production of antibodies by the immune system, after excessive exposure to these intracellular proteins, is a result of excessive or improper remodeling. By targeting the remodeling process it may be possible to lessen the effect autoantigens. Therefor, zacrp4 polypeptides, fragments, fusions, agonists; antagonists and the like would be beneficial in mediating a variety of autoimmune and remodeling diseases.

It is possible that an improper remodeling response to connective tissue or muscle injury in the joints results in sensitivity to excessive release of cellular components at the site of the injury. Zacrp6 polypeptides, fragments, fusions and the like would be useful in determining if an association exists between such a response and the inflammation associated with arthritis. Such indicators include a reduction in inflammation and relief of pain or stiffness. In animal models, indications would be derived from macroscopic inspection of joints and change in swelling of hind paws. Zacrp6 polypeptides, fragments, fusions and the like can be administered to animal models of osteoarthritis (Kikuchi et al., Osteoarthritis Cartilage 6:177-86, 1998 and Lohmander et al., Arthritis Rheum. 42:534-44, 1999) to look for inhibition of tissue destruction that results from inflammation stimulated by the action of collagenase.

Autoantigens diagnostic of scleroderma are to what would be consider cytoplasmic proteins. A knockout animal for zacrp4, as described herein, would be useful in determining if antibodies to zacrp4 proteins, fragments, fusions and the like are raised as a response to inflammation due to improper or incomplete repair of local tissue in response to stress.

Zacrp4 polypeptides, fragments, fusions and the like, as provided herein, would be useful in determining if excessive and/or inappropriate arterial remodeling plays a role in plaque formation in arterial sclerosis and arterial injury, such as arterial occlusion, using methods provided herein. Treatment of a vascular injury (and underlying extracellular matrix) with adipocyte complement protein zsig37 appears to alter the process of vascular remodeling at a very early stage (co-pending U.S. patent Ser. No. 09/253,604). Treatment with an adipocyte complement protein may act to keep platelets relatively quiescent after injury, eliminating excessive recruitment of pro-remodeling and proinflammatory proteins and cells.

Other members of the family may modulate remodeling induced by the presence of fat, or cholesterol for instance. Excessive amounts of cholesterol and fat in the blood might activate remodeling, in the absence of the correct adipocyte complement protein family member.

ACRP30 is expressed only in actively proliferating adipose tissue.

Connective tissue remodeling is tightly linked to this activation of fat cells. There is clearly a link between excessive weight gain (fat) and diabetes. It is therefore likely that ACRP30 is involved in fat remodeling and this process is overtaxed in obese individuals. As a result, the effects of improper and inadequate fat storage contribute to the onset of Type II diabetes.

Zacrp4 polypeptides, fragments, fusion proteins, antibodies, agonists or antagonists of the present invention can be used in methods for promoting blood flow within the vasculature of a mammal by reducing the number of platelets that adhere and are activated and the size of platelet aggregates. Used to such an end, Zacrp4 can be administered prior to, during or following an acute vascular injury in the mammal. Vascular injury may be due to vascular reconstruction, including but not limited to, angioplasty, coronary artery bypass graft, microvascular repair or anastomosis of a vascular graft. Also contemplated are vascular injuries due to trauma stroke or aneurysm. In other preferred methods the vascular injury is due to plaque rupture, degradation of the vasculature, complications associated with diabetes and atherosclerosis. Plaque rupture in the coronary artery induces heart attack and in the cerebral artery induces stroke. Use of zacrp4 polypeptides, fragments, fusion proteins, antibodies, agonists or antagonists in such methods would also be useful for ameliorating whole system diseases of the vasculature associated with the immune system, such as disseminated intravascular coagulation (DIC) and SIDs. Additionally the complement inhibiting activity would be useful for treating non-vasculature immune diseases such as arteriolosclerosis.

A correlation has been found between the presence of C1q in localized ischemic myocardium and the accumulation of leukocytes following coronary occlusion and reperfusion. Release of cellular components following tissue damage triggers complement activation which results in toxic oxygen products that may be the primary cause of myocardial damage (Rossen et al., Circ. Res. 62:572-84, 1998 and Tenner, ibid.). Blocking the complement pathway was found to protect ischemic myocardium from reperfusion injury (Buerke et al., J. Pharm. Exp. Therp. 286:429-38, 1998). Proteins having complement inhibition and C1q binding activity would be useful for such purposes.

Complement and C1q play a role in inflammation. The complement activation is initiated by binding of C1q to immunoglobulins (Johnston, Pediatr. Infect. Dis. J. 12:933-41, 1993; Ward and Ghetie, Therap. Immunol. 2:77-94, 1995). Inhibitors of C1q and complement would be useful as anti-inflammatory agents. Such application can be made to prevent infection. Additionally, such inhibitors can be administrated to an individual suffering from inflammation mediated by complement activation and binding of immune complexes to C1q. Inhibitors of C1q and complement would be useful in methods of mediating wound repair, enhancing progression in wound healing by overcoming impaired wound healing. Progression in wound healing would include, for example, such elements as a reduction in inflammation, fibroblasts recruitment, wound retraction and reduction in infection.

Radiation hybrid mapping is a somatic cell genetic technique developed for constructing high-resolution, contiguous maps of mammalian chromosomes (Cox et al., Science 250:245-50, 1990). Partial or full knowledge of a gene's sequence allows the designing of PCR primers suitable for use with chromosomal radiation hybrid mapping panels. Commercially available radiation hybrid mapping panels which cover the entire human genome, such as the Stanford G3 RH Panel and the GeneBridge 4 RH Panel (Research Genetics, Inc., Huntsville, Ala.), are available. These panels enable rapid, PCR based, chromosomal localizations and ordering of genes, sequence-tagged sites (STSs), and other nonpolymorphic- and polymorphic markers within a region of interest. This includes establishing directly proportional physical distances between newly discovered genes of interest and previously mapped markers. The precise knowledge of a gene's position can be useful in a number of ways including: 1) determining if a sequence is part of an existing contig and obtaining additional surrounding genetic sequences in various forms such as YAC-, BAC- or cDNA clones, 2) providing a possible candidate gene for an inheritable disease which shows linkage to the same chromosomal region, and 3) for cross-referencing model organisms such as mouse which may be beneficial in helping to determine what function a particular gene might have.

The gene encoding the zacrp4 polypeptide was mapped to human chromosome 1, region q11. The present invention also provides reagents which will find use in diagnostic applications. For example, the zacrp4 gene, a probe comprising zacrp4 DNA or RNA, or a subsequence thereof can be used to determine if the zacrp4 gene is present on chromosome 11 or if a mutation has occurred. Detectable chromosomal aberrations at the zacrp4 gene locus include, but are not limited to, aneuploidy, gene copy number changes, insertions, deletions, restriction site changes and rearrangements. These aberrations can occur within the coding sequence, within introns, or within flanking sequences, including upstream promoter and regulatory regions, and may be manifested as physical alterations within a coding sequence or changes in gene expression level.

In general, these diagnostic methods comprise the steps of (a) obtaining a genetic sample from a patient; (b) incubating the genetic sample with a polynucleotide probe or primer as disclosed above, under conditions wherein the polynucleotide will hybridize to complementary polynucleotide sequence, to produce a first reaction product; and (iii) comparing the first reaction product to a control reaction product. A difference between the first reaction product and the control reaction product is indicative of a genetic abnormality in the patient. Genetic samples for use within the present invention include genomic DNA, cDNA, and RNA. The polynucleotide probe or primer can be RNA or DNA, and will comprise a portion of SEQ ID NO:1, the complement of SEQ ID NO:1, or an RNA equivalent thereof. Suitable assay methods in this regard include molecular genetic techniques known to those in the art, such as restriction fragment length polymorphism (RFLP) analysis, short tandem repeat (STR) analysis employing PCR techniques, ligation chain reaction (Barany, PCR Methods and Applications 1:5-16, 1991), ribonuclease protection assays, and other genetic linkage analysis techniques known in the art (Sambrook et al., ibid.; Ausubel et. al., ibid.; Marian, Chest 108:255-65, 1995). Ribonuclease protection assays (see, e.g., Ausubel et al., ibid., ch. 4) comprise the hybridization of an RNA probe to a patient RNA sample, after which the reaction product (RNA-RNA hybrid) is exposed to RNase. Hybridized regions of the RNA are protected from digestion. Within PCR assays, a patient's genetic sample is incubated with a pair of polynucleotide primers, and the region between the primers is amplified and recovered. Changes in size or amount of recovered product are indicative of mutations in the patient. Another PCR-based technique that can be employed is single strand conformational polymorphism (SSCP) analysis (Hayashi, PCR Methods and Applications 1:34-8, 1991).

For pharmaceutical use, the proteins of the present invention can be formulated with pharmaceutically acceptable carriers for parenteral, oral, nasal, rectal, topical, transdermal administration or the like, according to conventional methods. In general, pharmaceutical formulations will include a zacrp4 protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton Pa., 19^{th} ed., 1995. Therapeutic doses will generally be determined by the clinician according to accepted standards, taking into account the nature and severity of the condition to be treated, patient traits, etc. Determination of dose is within the level of ordinary skill in the art.

As used herein a “pharmaceutically effective amount” of a zacrp4 polypeptide, fragment, fusion protein, agonist or antagonist is an amount sufficient to induce a desired biological result. The result can be alleviation of the signs, symptoms, or causes of a disease, or any other desired alteration of a biological system. For example, an effective amount of a zacrp4 polypeptide is that which provides either subjective relief of symptoms or an objectively identifiable improvement as noted by the clinician or other qualified observer. Such an effective amount of a zacrp4 polypeptide would provide, for example, recovery from microbial infection, improved cognitive function, changes in metabolic function that result in weight modification or a decrease in glucose levels in patients with IDDM. Effective amounts of the zacrp4 polypeptides can vary widely depending on the disease or symptom to be treated. The amount of the polypeptide to be administered and its concentration in the formulations, depends upon the vehicle selected, route of administration, the potency of the particular polypeptide, the clinical condition of the patient, the side effects and the stability of the compound in the formulation. Thus, the clinician will employ the appropriate preparation containing the appropriate concentration in the formulation, as well as the amount of formulation administered, depending upon clinical experience with the patient in question or with similar patients. Such amounts will depend, in part, on the particular condition to be treated, age, weight, and general health of the patient, and other factors evident to those skilled in the art. Typically a dose will be in the range of 0.01-100 mg/kg of subject. In applications such as balloon catheters the typical dose range would be 0.05-5 mg/kg of subject. Doses for specific compounds may be determined from in vitro or ex vivo studies in combination with studies on experimental animals. Concentrations of compounds found to be effective in vitro or ex vivo provide guidance for animal studies, wherein doses are calculated to provide similar concentrations at the site of action.

Polynucleotides encoding zacrp4 polypeptides are useful within gene therapy applications where it is desired to increase or inhibit zacrp4 activity. If a mammal has a mutated or absent zacrp4 gene, the zacrp4 gene can be introduced into the cells of the mammal. In one embodiment, a gene encoding a zacrp4 polypeptide is introduced in vivo in a viral vector. Such vectors include an attenuated or defective DNA virus, such as, but not limited to, herpes simplex virus (HSV), papillomavirus, Epstein Barr virus (E13V), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zacrp4 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; WIPO Publication WO 95/07358; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zacrp4 gene transcription, such as to inhibit cell proliferation in vivo. Polynucleotides that are complementary to a segment of a zacrp4-encoding polynucleotide (e.g., a polynucleotide as set froth in SEQ ID NO: 1) are designed to bind to zacrp4-encoding mRNA and to inhibit translation of such mRNA. Such antisense polynucleotides are used to inhibit expression of zacrp4 polypeptide-encoding genes in cell culture or in a subject.

Transgenic mice, engineered to express the zacrp4 gene, and mice that exhibit a complete absence of zacrp4 gene function, referred to as “knockout mice” (Snouwaert et al., Science 257:1083, 1992), may also be generated (Lowell et al., Nature 366:740-42, 1993). These mice may be employed to study the zacrp4 gene and the protein encoded thereby in an in vivo system.

Polynucleotides and polypeptides of the present invention are also useful as educational tools for use in laboratory practicum kits for academic courses related to genetics and molecular biology, protein chemistry and antibody production and analysis. Due to its unique polynucleotide and polypeptide sequence, molecules of zacrp4 can be used as standards or as “unknowns” for testing purposes. For example, zacrp4 polynucleotides can be used as an aid to teach a student how to prepare expression constructs for bacterial, viral, and/or mammalian expression, including fusion constructs, wherein zacrp4 is the gene to be expressed; for determining the restriction endonuclease cleavage sites of the polynucleotides; determining mRNA and DNA localization of zacrp4 polynucleotides in tissues (i.e., by Northern and Southern blotting as well as polymerase chain reaction); and for identifying related polynucleotides and polypeptides by nucleic acid hybridization.

Zacrp4 polypeptides can also be used as an educational aid to teach preparation of antibodies; identification of proteins by Western blotting; protein purification; determination of the weight of expressed zacrp4 polypeptides as a ratio to total protein expressed; identification of peptide cleavage sites; coupling amino and carboxyl terminal tags; amino acid sequence analysis, as well as, but not limited to, monitoring biological activities of both the native and tagged protein (i.e., receptor binding, signal transduction, proliferation, and differentiation) in vitro and in vivo. Antibodies that bind specifically to zacrp-4 can be used as an aid to instruct students how to prepare affinity chromatography columns to purify zacrp-4, and as a practicum for teaching a student how to design humanized antibodies.

Zacrp4 polypeptides can also be used to teach analytical skills such as mass spectrometry, circular dichroism to determine conformation, x-ray crystallography to determine the three-dimensional structure in atomic detail, nuclear magnetic resonance spectroscopy to reveal the structure of proteins in solution. For example, a kit containing zacrp4 can be given to the student to analyze as an unknown protein to developanalytical skills of the student or as a test of those skills. Since every polypeptide is unique, the educational utility of zacrp-4 would be unique unto itself.

The invention provides zacpr-4 gene, polypeptide or antibody packaged in educational kits to be used in academic programs allowing students to gain skill in art of molecular biology. Since each gene and protein is unique, each gene and protein provides unique challenges and learning experiences for students in a lab practicum setting.

The invention is further illustrated by the following non-limiting examples.

### EXAMPLES

### Example 1

### Identification of Zacrp4

Novel zacrp4 polynucleotides and polypeptides of the present invention were initially identified querying an expressed sequence tag (EST) database for proteins having homology to adipocyte complement related proteins. To identify the corresponding cDNA sequence, a clone was isolated from an arrayed human pituitary cDNA/plasmid library. The library was screened by PCR using oligonucleotides ZC20,839 (SEQ ID NO:5) and ZC20,840 (SEQ ID NO:6). Thermocycler conditions were as follows: 1 cycle at 94° C. for 2 minutes 30 seconds, followed by 30 cycles at 94° C. for 10 seconds, 64° C. for 20 seconds, 72° C. for 30 seconds, ending with a 7 minute extension at 72° C. The library was deconvoluted down to a positive pool of 250 clones. E. coli DH10B cells (GIBCO BRL, Gaithersburg, Md.) were transformed with this pool by electroporation following manufacturer's protocol. The transformed culture was titered and arrayed into a 96 well plate at ˜20 cells/well. The cells were grown overnight at 37° C. in LB+ampicillin. An aliquot of the cells were pelleted and screened using PCR to identify positive wells using oligonucleotide primers and PCR conditions were as described above. The remaining cells in the positive wells were plated and colonies screened by PCR to identify a single positive clone. The clone was subjected to sequence analysis using a Hybaid OmniGene Temperature Cycling System (National Labnet Co., Woodbridge, N.Y.). SequencherTM 3.0 sequence analysis software (Gene Codes Corporation, Ann Arbor, Mich.) was used for data analysis. The resulting 1196 bp sequence is disclosed in SEQ ID NO: 1.

### Example 2

### Tissue Distribution

Northerns were performed using Human Multiple Tissue Blots (MTN1, MTN2 and MTN3) from Clontech (Palo Alto, Calif.) were probed to determine the tissue distribution of human zacrp4. A clone described above was used as a template for the generation of a 303 bp cDNA probe based on the initially discovered EST sequence (SEQ ID NO:7) using the PCR. Oligo nucleotides ZC20,839 (SEQ ID NO:5) and ZC20,840 (SEQ ID NO:6) were used as primers. The probe was purified using a Gel Extraction Kit (Qiagen, Chatsworth, Calif.) according to manufacturer's instructions. The probe was then radioactively labeled using a Rediprime II DNA labeling kit (Amersham, Arlington Heights, Ill.) according to the manufacturer's specifications. The probe was purified using a NUCTrap push column (Stratagene Cloning Systems, La Jolla, Calif.). ExpressHyb (Clontech) solution was used for prehybridization and as a hybridizing solution for the Northern blots. Hybridization took place overnight at 55° C., using 1.5×10⁶ cpm/ml labeled probe. The blots were then washed in 2×SSC and 0.1% SDS at room temperature, then with 2×SSC and 0.1% SDS at 65° C., followed by a wash in 0.1×SSC and 0.1% SDS at 65° C. A single transcript of approximately 1.4 kb was seen in brain, spinal cord, ovary, and testis. Fainter signals were detected in thyroid, adrenal gland, bone marrow, small intestine, prostate, liver and colon.

A RNA Master Dot Blot (Clontech) that contained RNAs from various tissues that were normalized to 8 housekeeping genes were also probed and hybridized as described above. Expression was seen in all brain tissues and in fetal brain, spinal cord, ovary and pituitary gland. Fainter signals were detected in testis, uterus, prostate, salivary gland, lymph node and fetal liver and lung.

### Example 3

### Chromosomal Assignment and Placement of Zacrp4

Zacrp4 was mapped to chromosome 11 using the commercially available GeneBridge 4 Radiation Hybrid Panel (Research Genetics, Inc., Huntsville, Ala.). The GeneBridge 4 Radiation Hybrid Panel contains PCRable DNAs from each of 93 radiation hybrid clones, plus two control DNAs (the HFL donor and the A23 recipient). A publicly available WWW server (http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of zacrp4 with the GeneBridge 4 RH Panel, 20 μl reactions were set up in a 96-well microtiter plate (Stratagene, La Jolla, Calif.) and used in a “RoboCycler Gradient 96” thermal cycler (Stratagene). Each of the 95 PCR reactions consisted of 2 μl 10×KlenTaq PCR reaction buffer (Clontech Laboratories, Inc., Palo Alto, Calif.), 1.6 μl dNTPs mix (2.5 mM each, Perkin-Elmer, Foster City, Calif.), 1 μl sense primer, ZC 22,162, (SEQ ID NO:8), 1 μl antisense primer, ZC 22,168 (SEQ ID NO:9), 2 μl RediLoad (Research Genetics, Inc.), 0.4 μl 50×Advantage KlenTaq Polymerase Mix (Clontech Laboratories, Inc.), 25 ng of DNA from an individual hybrid clone or control and ddH₂O for a total volume of 20 μl. The reactions were overlaid with an equal amount of mineral oil and sealed. The PCR cycler conditions were as follows: an initial 1 cycle 5 minute denaturation at 94° C., 40 cycles of a 45 seconds denaturation at 94° C., 45 seconds annealing at 66° C. and 1 minute and 15 seconds extension at 72° C., followed by a final 1 cycle extension of 7 minutes at 72° C. The reactions were separated by electrophoresis on a 2% agarose gel (Life Technologies, Gaithersburg, Md.).

The results showed that zacrp4 maps 16.37 cR_{—}3000 from the framework marker D11S1350 on the chromosome 11 WICGR radiation hybrid map. Proximal and distal framework markers were D11S1350 and CHLC.GATA6G03, respectively. The use of surrounding markers positions Zacrp4 in the 11q11 region on the integrated LDB chromosome 11 map (The Genetic Location Database, University of Southhampton, WWW server: http://cedar.genetics.soton.ac.uk/public_html/).

What is claimed is:

1. An isolated polypeptide comprising:
a first C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2, and a cysteine residue corresponding to residue 70 of SEQ ID NO:2; and
a second C1q domain joined to the carboxy terminal of said first C1q domain, said second C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2, and a cysteine residue corresponding to residue 223, a glycine residue corresponding to residue 228 of SEQ ID NO:2.

2. An isolated polypeptide according to claim 1, further comprising a secretory signal sequence.

3. An isolated polypeptide according to claim 2, where said secretory signal sequence comprises amino acid residues 1-16 of SEQ ID NO:2

4. An isolated polypeptide comprising a sequence of amino acid residues that is at least 80% identical in amino acid sequence to residues 17 to 329 of SEQ ID NO:2, wherein said sequence comprises:
a first C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2, and a cysteine residue corresponding to residue 70 of SEQ ID NO:2; and
a second C1q domain joined to the carboxy terminal of said first C1q domain, said second C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2, and a cysteine residue corresponding to residue 223, a glycine residue corresponding to residue 228 of SEQ ID NO:2.

5. An isolated polypeptide according to claim 4, wherein said polypeptide is at least 90% identical in amino acid sequence to residues 17 to 329 of SEQ ID NO:2.

6. An isolated polypeptide of claim 5, wherein the amino acid percent identity is determined using a FASTA program with ktup=1, gap opening penalty=10, gap extension penalty=1, and substitution matrix=blosum62, with other parameters set as default.

7. An isolated polypeptide according to claim 5, wherein any differences between said polypeptide and SEQ ID NO:2 are due to conservative amino acid substitutions.

8. An isolated polypeptide according to claim 5, wherein said polypeptide specifically binds with an antibody that specifically binds with a polypeptide consisting of the amino acid sequence of SEQ ID NO:2.

9. An isolated polypeptide according to claim 5, wherein said first C1q domain comprises amino acid residues 17-159 of SEQ ID NO:2.

10. An isolated polypeptide according to claim 5, wherein said second C1q domain comprises amino acid residues 160-328 of SEQ ID NO:2.

11. An isolated polypeptide according to claim 5, wherein said polypeptide comprises residues 17-328 of SEQ ID NO:2.

12. An isolated polypeptide according to claim 4, covalently linked at the amino or carboxyl terminus to a moiety selected from the group consisting of affinity tags, toxins, radionucleotides, enzymes and fluorophores.

13. An isolated polypeptide comprising:
a signal sequence;
a first C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2 and a cysteine residue corresponding to amino acid residue 70 of SEQ ID NO:2; and
a second C1q domain joined to the carboxy terminal of said first C1q domain, said second C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2 and a cysteine residue corresponding to residue 223 of SEQ ID NO:2;
wherein said polypeptide specifically binds with an antibody that specifically binds with a polypeptide consisting of the amino acid sequence of SEQ ID NO:2.

14. An isolated polypeptide selected from the group consisting of:
a) a polypeptide consisting of amino acid residue 17 to amino acid residue 159 of SEQ ID NO:2 or
b) a polypeptide consisting of amino acid residue 160 to amino acid residue 329 of SEQ ID NO:2.

15. A fusion protein consisting essentially of a first portion and a second portion joined by a peptide bond, said first portion consisting of a polypeptide selected from the group consisting of:
a) polypeptide according the claim 1;
b) a polypeptide comprising the amino acid sequence of residues 17-159 of SEQ ID NO:2;
c) a polypeptide comprising the amino acid sequence of residues 160-329 of SEQ ID NO:2;
d) a polypeptide comprising the amino acid sequence of residues 17-329 of SEQ ID NO:2; and
said second portion comprising another polypeptide.

16. A polypeptide according to claim 1; in combination with a pharmaceutically acceptable vehicle.

17. A method of producing an antibody to a polypeptide comprising:
inoculating an animal with a polypeptide selected from the group consisting of:
a) polypeptide according to claim 1;
b) a polypeptide comprising the amino acid sequence of residues 17-159 of SEQ ID NO:2;
c) a polypeptide comprising the amino acid sequence of residues 160-329 of SEQ ID NO:2;
d) a polypeptide comprising the amino acid sequence of residues 17-329 of SEQ ID NO:2; and
e) a polypeptide or polypeptide fragment of SEQ ID NO:2; and
wherein said polypeptide elicits an immune response in the animal to produce the antibody; and
isolating the antibody from the animal.

18. An antibody or antibody fragment that specifically binds to a polypeptide according to claim 1.

19. An antibody according to claim 18, wherein said antibody is selected from the group consisting of:
a) polyclonal antibody;
b) murine monoclonal antibody;
c) humanized antibody derived from b); and
d) human monoclonal antibody.

20. An antibody fragment according to claim 18, wherein said antibody fragment is selected from the group consisting of F(ab′), F(ab), Fab′, Fab, Fv, scFv, and minimal recognition unit.

21. An anti-idiotype antibody that specifically binds to said antibody of claim 18.

22. An isolated polynucleotide molecule encoding a polypeptide according to claim 1.

23. An isolated polynucleotide molecule according to claim 22, wherein said polypeptide further comprises a secretory signal sequence.

24. An isolated polynucleotide molecule according to claim 23, where said secretory signal sequence comprises amino acid residues 1-16 of SEQ ID NO:2

25. An isolated polynucleotide molecule encoding a polypeptide according to claim 4.

26. An isolated polynucleotide molecule according to claim 25, wherein said polypeptide is at least 90% identical in amino acid sequence to residues 17-329 of SEQ ID NO:2.

27. An isolated polynucleotide molecule of claim 26, wherein the amino acid percent identity is determined using a FASTA program with ktup=1, gap opening penalty=10, gap extension penalty=1, and substitution matrix=blosum62, with other parameters set as default.

28. An isolated polynucleotide molecule according to claim 26, wherein any differences between said polypeptide and SEQ ID NO:2 are due to conservative amino acid substitutions.

29. An isolated polynucleotide molecule according to claim 26, wherein said polypeptide specifically binds with an antibody that specifically binds with a polypeptide consisting of the amino acid sequence of SEQ ID NO:2.

30. An isolated polynucleotide molecule according to claim 26, wherein said polynucleotide molecule remains hybridized following stringent wash conditions to a polynucleotide consisting of the nucleotide sequence of SEQ ID NO: 1, or the complement of SEQ ID NO:1.

31. An isolated polynucleotide molecule according to claim 26, wherein said first C1q domain comprises amino acid residues 17-159 of SEQ ID NO:2.

32. An isolated polynucleotide molecule according to claim 26, wherein said second C1q domain comprises amino acid residues 160-329 of SEQ ID NO:2.

33. An isolated polynucleotide molecule according to claim 26, wherein said polypeptide comprises residues 17-329 of SEQ ID NO:2.

34. An isolated polynucleotide molecule encoding a polypeptide comprising:
a signal sequence;
a first carboxyl-terminal C1q domain comprising a sequence of SEQ ID NO:3, 10 beta strands corresponding to amino acid residues 31-35, 52-54, 60-63, 67-69, 73-84, 89-95, 101-108, 112-126, 131-136 and 150-154 of SEQ ID NO:2 and a cysteine residue corresponding to amino acid residue 70 of SEQ ID NO:2; and
a second carboxyl-terminal C1q domain comprising a sequence of SEQ ID NO:3, 10 beta strands corresponding to amino acid residues 179-183, 206-208, 214-217, 221-223, 227-238, 243-249, 254-262, 267-278, 283-288 and 305-309 of SEQ ID NO:2 and a cysteine residue corresponding to residue 223 of SEQ ID NO:2;
wherein said polypeptide specifically binds with an antibody that specifically binds with a polypeptide consisting of the amino acid sequence of SEQ ID NO:2, and
said polynucleotide molecule remains hybridized following stringent wash conditions to a polynucleotide consisting of the nucleotide sequence of SEQ ID NO: 1, or the complement of SEQ ID NO:1.

35. An isolated polynucleotide molecule selected from the group consisting of,
a) a contiguous sequence of nucleotides from nucleotide 1 to nucleotide 1357 of SEQ ID NO:1;
b) a contiguous sequence of nucleotides from nucleotide 210 to nucleotide 1196 of SEQ ID NO:1;
c) a contiguous sequence of nucleotides from nucleotide 258 to nucleotide 1196 of SEQ ID NO:1;
d) a contiguous sequence of nucleotides from nucleotide 258 to nucleotide 686 of SEQ ID NO:1;
e) a polynucleotide encoding a polypeptide consisting of the sequence of amino acid residues 17 to 159 of SEQ ID NO:2;
f) a polynucleotide encoding a polypeptide consisting of the sequence of amino acid residues 160 to 329 of SEQ ID NO:2;
g) a polynucleotide encoding a polypeptide consisting of the sequence of amino acid residues 17 to 329 of SEQ ID NO:2;
h) a polynucleotide that remains hybridized following stringent wash conditions to a polynucleotide consisting of the nucleotide sequence of SEQ ID NO:1, or the complement of SEQ ID NO: 1;
i) the nucleotide sequences complementary to a), b), c), d), e), f), g) or h) and m) degenerate nucleotide sequences of e), f) or g).

36. An isolated polynucleotide molecule encoding a fusion protein consisting essentially of a first portion and a second portion joined by a peptide bond, said first portion is selected from the group consisting of:
a) a polypeptide according to claim 1;
b) a polypeptide comprising the amino acid sequence of residues 17-159 of SEQ ID NO:2;
c) a polypeptide comprising the amino acid sequence of residues 160-329 of SEQ ID NO:2; and
d) a polypeptide comprising the amino acid sequence of residues 17-329 of SEQ ID NO:2; and
said second portion comprising another polypeptide.

37. An isolated polynucleotide molecule consisting of the sequence of nucleotide 1 to nucleotide 987 of SEQ ID NO:4.

38. An expression vector comprising the following operably linked elements:
a transcription promoter;
a DNA segment encoding a polypeptide according to claim 1; and
a transcription terminator.

39. An expression vector according to claim 38, wherein said DNA segment encodes a polypeptide covalently linked at the amino or carboxyl terminus to an affinity tag.

40. An expression vector according to claim 38, wherein said DNA segment further encodes a secretory signal sequence operably linked to said polypeptide.

41. An expression vector according the claim 40, wherein said secretory signal sequence comprises residues 1-16 of SEQ ID NO:2.

42. A cultured cell into which has been introduced an expression vector according to claim 38, wherein said cell expresses said polypeptide encoded by said DNA segment.

43. A method of producing a protein comprising:
culturing a cell into which has been introduced an expression vector according to claim 38;
whereby said cell expresses said polypeptide encoded by said DNA segment; and
recovering said expressed protein.
